(19) United States
(12) Patent Application Publication
(10) Pub. No.: US 2014/0345640 A1
(43) Pub. Date: Nov. 27, 2014

(54) Powdery Hair Cosmetics

(71) Applicant: Henkel AG & Co. KGaA, Dusseldorf (DE)

(72) Inventors: Thorsten Knappe, Schenefeld (DE); Anna Henschel, Winsen (Luhe) (DE)

(73) Assignee: Henkel AG & Co. KGaA, Dusseldorf (DE)

(21) Appl. No.: 14/364,595

(22) PCT Filed: Nov. 9, 2012
(86) PCT No.: PCT/EP2012/072254

§ 371 Date: Jun. 11, 2014

(30) Foreign Application Priority Data
Dec. 16, 2011 (DE) 10 2011 088 815.2

(51) Int. Cl. A61K 8/02; A61K 8/81; A61Q 5/06; A61K 8/25; A61K 8/92; A61K 8/891; A61K 8/86

(52) U.S. Cl. CPC A61K 8/022; A61K 8/891; A61K 8/817; A61K 8/86; A61K 8/25; A61K 8/927; A61K 8/922; A61Q 5/06; A61K 2800/20
USPC 132/203; 424/401; 424/70.12; 424/70.15; 424/70.11

(57) ABSTRACT

Powdery compositions contain at least one particulate and amorphous metal oxide, with the proviso that at least one cosmetic active ingredient is present in said metal oxide in absorbed form, said active ingredient being selected from waxy esters, strengthening polymers, film-forming polymers, oxidation dye precursors, anionic surfactants, keratin-reducing compounds or mixtures thereof. Said powdery compositions are highly suitable for temporarily shaping keratin-containing fibers, especially human hair. Hair treated with said powders can be strengthened and retains its natural gloss.

### CROSS REFERENCE TO RELATED APPLICATIONS

The present disclosure is a U.S. National Stage entry under 35 U.S.C. §371 based on International Application No. PCT/EP2012/072254, filed Nov. 9, 2012 which was published under PCT Article 21(2) and which claims priority to German Patent Application No. 10 2011 088 815.2 filed on Dec. 16, 2011, which is incorporated herein by reference in its entirety.

### TECHNICAL FIELD

The technical field relates to the utilization of powdered compositions for the cosmetic treatment of keratinic fibers, in particular human hair.

### BACKGROUND

Powdered cosmetics have long been known to one skilled in the art. They have already been used for some time, for example, in the skin treatment sector. Typical examples are make-up powder or eye shadow. Surfactant-free but solvent-containing dry shampoos, which are sprayed onto the fibers by means of an aerosol apparatus, have also long been used for hair treatment purposes. The starch particles contained in the dry shampoo absorb sebum and dirt once the solvent has evaporated, and are brushed out after a contact time. One skilled in the art is also familiar with powdered hair colors, although these are not applied in powder form onto the fibers but instead are stirred with separately added water to form a coloring cream. The resulting cream is then utilized on the hair.

Styling agents for the deformation of keratinic fibers have generally been known for some time, and are employed in a variety of configurations to build up, refresh, and retain hairstyles that, for many hair types, can be obtained only using setting active agents. An important role is played here both by hair treatment agents that serve for permanent shaping of the hair, and by those that serve for temporary shaping. Temporary shaping results that are intended to produce good hold without negatively affecting the healthy appearance of the hair, for example its shine, can be obtained, for example, by means of hair sprays, hair waxes, hair gels, blow-dry waves, etc.

Corresponding agents for temporary shaping usually contain synthetic polymers as a shaping component. Preparations that contain a polymer can be applied onto the hair by means of propellant gases or using a pump mechanism. Hair gels and hair waxes in particular, on the other hand, are as a rule not applied directly onto the hair but instead distributed in the hair using a comb or one's hands.

Known forms of temporary styling agents often cannot be metered with satisfactory accuracy. Hair gels, hair creams, and hair waxes, for example, are difficult to distribute once they have been applied onto the hair. As soon as the comb or the hands onto which the styling agent has been applied come into contact with the first hair areas, comparatively large quantities of styling agent are delivered onto the hair. Relatively little styling agent, on the other hand, is incorporated into hair areas that are reached only later with the comb or hands. The consequence of this is that the user either must from the outset apply a large quantity of styling agent so that even those hair areas reached last receive sufficient styling agent, or is forced to apply the styling agent in multiple steps, different hair areas being treated in each case. Hair sprays can be distributed more uniformly onto the hair. But because the user has no ability to visually perceive the total quantity of styling agent applied, the risk exists that more styling agent that would actually be necessary is applied onto the hair.

The document WO 2007/051511 A1 discloses the use of a powdered composition, containing 50 to 95 wt % of an aqueous solvent, hydrophobized silicon dioxide powder, and at least a film-forming and/or setting polymer that is present in the aqueous solvent, for the temporary deformation of keratinic fibers.

The document WO 2010/054980 A1 discloses the use of a powdered composition having core-shell particles for the temporary deformation of keratinic fibers, wherein the shell of the particles contains at least one hydrophobized metal oxide powder and the core comprises a liquid aqueous phase. These powdered core-shell particles comprise at least one film-forming and/or setting polymer in the form of particles.

The document WO-A1-2011/076518 describes powdered compositions that can be converted into a liquid and that comprise an active substance sorbed onto a carrier.

The powdered compositions known from the existing art exhibit a limited utilization profile in particular in the field of hair cosmetics.

Accordingly, it is desirable to provide a powdered composition for the cosmetic treatment of keratinic fibers that, in the context of all embodiments,
can be metered accurately and simply,
can be effectively distributed on the keratin-containing fibers without clumping,
as a leave-on cosmetic (i.e., the cosmetic is not rinsed/washed off the substrate after application), avoids a visible matte effect on the fibers (but could nevertheless be used as a matting composition by the addition of suitable ingredients),
as a rinse-off cosmetic (i.e., the cosmetic is rinsed/washed off the substrate after application), can readily be rinsed/washed off the fibers,
has improved shelf stability, and
delivers cosmetic active agents quickly and reliably onto the substrate.

It also is desirable to provide a powdered composition with suitability for the deformation of keratinic fibers, in particular human hair, that is compatible with the waxy esters that are usual in the hair-styling sector, and stable. When the waxy-ester-containing powdered composition is utilized, the powder is capable of transforming into a liquid to pasty form or consistency. These powdered compositions are employed predominantly as a leave-on cosmetic.

It is further desirable to provide a powdered composition with suitability for the deformation of keratinic fibers, in particular human hair, that does not form fine dusts of setting polymers contained in the composition as a solid. In addition, it is desirable that the setting polymer is readily able to be released from the powdered composition and be distributed uniformly onto the substrate. These powdered compositions are employed predominantly as a leave-on cosmetic.

In addition, it is desirable to provide a powdered composition with suitability for caring for keratinic fibers, in particular human hair, that does not form fine dusts of film-forming polymers contained in the composition as a solid. In addition, the setting polymer is intended to be readily able to be released from the powdered composition and be distributed uniformly onto the substrate.

It also is desirable to provide a powdered composition with suitability for cleaning keratinic fibers, in particular human hair, in the presence of aqueous and powdered core-shell particles the latter that remains stable even in the presence of at least about 2 wt % surfactants having washing activity. These powdered compositions are furnished predominantly as a rinse-off cosmetic.

It is further desirable to provide a powdered composition with suitability for oxidative hair coloring that can be stored and utilized as two components reactive with another (i.e. oxidation dye precursor(s) and oxidizing agent(s)) in the same powdered composition. These powdered compositions are furnished predominantly as a rinse-off cosmetic.

### SUMMARY

Powdered compositions and methods for cosmetic treatment of keratin-containing fibers are provided. In accordance with an exemplary embodiment, a powdered composition comprises at least one particulate amorphous metal oxide, with the provision that at least one cosmetic active agent that is chosen from waxy esters, setting polymers, film-forming polymers, oxidation dye precursors, anionic surfactants, keratin-reducing compounds, and mixtures thereof is present in a manner sorbed in said metal oxide.

In another exemplary embodiment, a method for cosmetic treatment of keratin-containing fibers, in particular human hair, is provided. The method includes:
a) optionally moistening the keratin-containing fibers;
b) applying a powdered composition onto the keratin-containing fibers, the powdered composition comprising:
   at least one particulate amorphous metal oxide, with the provision that at least one cosmetic active agent that is chosen from waxy esters, setting polymers, film-forming polymers, oxidation dye precursors, anionic surfactants, keratin-reducing compounds, and mixtures thereof is present in a manner sorbed in said metal oxide;
c) exposing the powdered composition, during or after application onto the keratin-containing fibers, to a mechanical load, if the powdered composition comprises core-shell particles whose shell contains particles of at least one hydrophobized metal oxide powder and whose core comprises a liquid aqueous phase, whereby the powdered composition converts into a liquid, and
d) working the applied composition into the keratin-containing fibers and optionally rinsing the composition from the keratin-containing fibers.

### DETAILED DESCRIPTION

In accordance with an exemplary embodiment, a powdered composition contains at least one particulate amorphous metal oxide, with the provision that at least one cosmetic active agent that is selected from waxy esters, setting polymers, film-forming polymers, oxidation dye precursors, anionic surfactants, keratin-reducing compounds, or mixtures thereof is present in a manner adsorbed in said metal oxide.

“Particles” as used herein are particles present in grain form (cf. DIN 66160: 1992-09) of solids. Compositions whose particles are freely pourable under their own weight (cf. DIN EN ISO 6186: 1998-08) are “powdered” for purposes herein.

An “amorphous state” of a solid exists when the atomic modules thereof are arranged predominantly not in crystal lattices. In contrast to a crystalline substance, in which in addition to a short-range order between the modules (i.e., constant distances and angles with respect to the closest adjacent atoms), a long-range order (regular repetition of a pattern in a crystal lattice) also exists, in the amorphous state only a more or less pronounced short-range order is present.

“Sorption” is the collective term for all processes (including absorption and adsorption) in which one substance is selectively taken up by another substance in contact with it. The particulate amorphous metal oxide is the sorbent; the cosmetic active agent is the sorbate.

When mixtures of the aforesaid cosmetic active agents are present, several different active agents can be sorbed together onto the same particle of the particulate amorphous metal oxide (combined sorption).

In addition, different active agents can be sorbed individually onto particles of an amorphous metal oxide. Thus only one active agent is present in a manner sorbed onto the particles. These particles are then mixed (separate sorption). What results therefrom is a powdered composition having an active-agent mixture sorbed onto particulate amorphous metal oxides, wherein only one of the cosmetic active agents is present in a manner sorbed onto each particle of that mixture.

In the context of a particularly preferred embodiment, the particulate amorphous metal oxide is a precipitated silicic acid. A precipitated silicic acid having a proportion from about 0.1 to about 1.0 wt % Na₂O, based on its weight, is in turn preferred as a precipitated silicic acid.

In a preferred embodiment, the particulate amorphous metal oxide has a DBP number from about 50 to about 800 g, preferably from about 100 to about 500 g, particularly preferably from about 200 to about 400 g, very particularly preferably from about 250 to about 350 g, in each case per 100 g of metal oxide. The DBP number is always indicated in g DBP (dibutyl phthalate) per 100 g of metal oxide, and is determined as defined by ASTM D 2414 based on dry substance.

Release of the sorbed cosmetic active agent occurs particularly well when the particulate amorphous metal oxide of the powdered composition contemplated herein produces, in a 5-wt % slurry in distilled water, a pH of the resulting mixture from about 4.5 to about 8.0, in particular from about 5.0 to about 7.5. This embodiment is particularly well suited for increasing the release of waxy esters and/or setting polymers and/or film-forming polymers.

It has furthermore been found that the powdered composition contemplated herein functions particularly well when the particulate amorphous metal oxide of the powdered composition has a BET surface area from about 50 to about 1000 m²/g, preferably from about 100 to about 750 m²/g, particularly preferably from about 150 to about 500 m²/g (measured in each case per ISO 5794-1).

A particularly preferred powdered composition comprises at least one particulate amorphous metal oxide of the embodiments (A) to (F):

(A):

A particulate amorphous metal oxide in the form of a precipitated silicic acid
having a BET surface area from about 50 to about 1000 m²/g, preferably from about 100 to about 750 m²/g, particularly preferably from about 150 to about 500 m²/g (measured in each case per ISO 5794-1), and
   with the provision that at least one cosmetic active agent that is selected from waxy esters, setting polymers, film-forming polymers, oxidation dye precursors, anionic surfactants, keratin-reducing compounds, or mixtures thereof is present in a manner sorbed in said metal oxide.

(B):

A particulate amorphous metal oxide in the form of a precipitated silicic acid
having a DBP number from about 50 to about 800 g, preferably from about 100 to about 500 g, particularly preferably from about 200 to about 400 g, very particularly preferably from about 250 to about 350 g, in each case per 100 g of metal oxide, and
   with the provision that at least one cosmetic active agent that is selected from waxy esters, setting polymers, film-forming polymers, oxidation dye precursors, anionic surfactants, keratin-reducing compounds, or mixtures thereof is present in a manner sorbed in said metal oxide.

(C):

A particulate amorphous metal oxide in the form of a precipitated silicic acid
having a BET surface area from about 50 to about 1000 m²/g, preferably from about 100 to about 750 m²/g, particularly preferably from about 150′ to about 500 m²/g (measured in each case per ISO 5794-1), and
having a DBP number from about 50′ to about 800 g, preferably from about 100 to about 500 g, particularly preferably from about 200 to about 400 g, very particularly preferably from about 250 to about 350 g, in each case per 100 g of metal oxide,
   with the provision that at least one cosmetic active agent that is selected from waxy esters, setting polymers, film-forming polymers, oxidation dye precursors, anionic surfactants, keratin-reducing compounds, or mixtures thereof is present in a manner sorbed in said metal oxide.

(D):

A particulate amorphous metal oxide in the form of a precipitated silicic acid
having a BET surface area from about 50 to about 1000 m²/g, preferably from about 100 to about 750 m²/g, particularly preferably from about 150 to about 500 m²/g (measured in each case per ISO 5794-1),
which produces, in a 5-wt % slurry in distilled water, a pH of the resulting mixture from about 4.5 to about 8.0, in particular from about 5.0 to about 7.5,
   with the provision that at least one cosmetic active agent that is selected from waxy esters, setting polymers, film-forming polymers, oxidation dye precursors, anionic surfactants, keratin-reducing compounds, or mixtures thereof is present in a manner sorbed in said metal oxide.

(E):

A particulate amorphous metal oxide in the form of a precipitated silicic acid
having a DBP number from about 50 to about 800 g, preferably from about 100 to about 500 g, particularly preferably from about 200 to about 400 g, very particularly preferably from about 250 to about 350 g, in each case per 100 g of metal oxide, and
which produces, in a 5-wt % slurry in distilled water, a pH of the resulting mixture from about 4.5 to about 8.0, in particular from about 5.0 to about 7.5,
   with the provision that at least one cosmetic active agent that is selected from waxy esters, setting polymers, film-forming polymers, oxidation dye precursors, anionic surfactants, keratin-reducing compounds, or mixtures thereof is present in a manner sorbed in said metal oxide.

(F):

A particulate amorphous metal oxide in the form of a precipitated silicic acid
having a BET surface area from about 50 to about 1000 m²/g, preferably from about 100 to about 750 m²/g, particularly preferably from about 150 to about 500 m²/g (measured in each case per ISO 5794-1),
having a DBP number from about 50 to about 800 g, preferably from about 100 to about 500 g, particularly preferably from about 200 to about 400 g, very particularly preferably from about 250 to about 350 g, in each case per 100 g of metal oxide, and
which produces, in a 5-wt % slurry in distilled water, a pH of the resulting mixture from about 4.5 to about 8.0, in particular from about 5.0 to about '7.5,
   with the provision that at least one cosmetic active agent that is selected from waxy esters, setting polymers, film-forming polymers, oxidation dye precursors, anionic surfactants, keratin-reducing compounds, or mixtures thereof is present in a manner adsorbed in said metal oxide.

It is generally preferred, and in turn preferred in particular in the context of the aforementioned preferred embodiments (in particular the embodiments (A) to (F)), if the aforesaid cosmetic active agent (or mixtures thereof) is present in a manner sorbed in the particulate amorphous metal oxide in a weight ratio range from about 1:1 to about 9:1, in particular from about 3:1 to about 4:1.

At least one waxy ester (also hereinafter called a “wax ester”) is suitable as a sorbed cosmetic active agent, in particular for provision of a powdered composition for the deformation of keratin-containing fibers. Waxy esters useful herein exhibit a melting point in a range from about 40° C. to about 95° C. at 1013 mbar.

The waxy esters are preferably selected from vegetable, animal, and mineral waxes wherein those waxes which have a melting point in the range from about 40° C. to about 90° C. are preferred.

Particularly preferably herein, the powdered composition contemplated herein contains at least one waxy ester selected from at least one wax of the group: beeswax (cera alba), carnauba wax, candelilla wax, montan wax, rice bran wax, hydrogenated vegetable oil, and cetyl palmitate.

In an exemplary embodiment, the combination of multiple waxes also can be used. An addition of carnauba wax, for example, can be used to raise the melting point and drop point of another wax and decrease its tackiness. These other waxes can be different from the waxy esters. Microcrystalline waxes, in particular microcrystalline paraffin wax and ozocerite wax, for example, are preferred. A number of wax mixtures, optionally mixed with further additives, are correspondingly available commercially. Those having the designations “Spezialwachs 7686 OE” (a mixture of cetyl palmitate, beeswax, microcrystalline wax, and polyethylene, having a melting range of 73 to 75° C.; manufacturer: Kahl & Co.) and “Weichceresin® FL 400” (a vaseline/vaseline oil/wax mixture having a melting point of 50 to 54° C.; manufacturer: Parafluid Mineralölgesellschaft) are examples of preferable mixtures.

It is preferred, in the context of all the waxy esters recited (in particular those mentioned as preferred), to use one of the preferred particulate amorphous metal oxides (in particular one of the embodiments (A) to (F)) (see above) for sorption of the waxy ester.

The powdered compositions contemplated herein contain the waxy esters preferably in quantities from about 50 wt % to about 90 wt % based on the entire agent. Quantities from about 75 to about 80 wt %, based on the entire agent, are particularly preferred.

In an exemplary embodiment, in the context of the waxy-ester-containing powdered compositions, a particulate amorphous metal oxide is present on whose particles in sum at least one waxy ester and at least one emulsifier agent are present in a sorbed manner. In the context of this embodiment, either the waxy ester and the emulsifier agent can be present in a manner sorbed together on the same particle of the particulate amorphous metal oxide (combined sorption), and/or what is present is a mixture of particles of a particulate amorphous metal oxide having a sorbed emulsifier agent with particles of a particulate amorphous metal oxide having a sorbed waxy ester (separate sorption).

In an embodiment, the emulsifier agent is a nonionic oil-in-water emulsifier agent.

The powdered compositions contemplated herein contain emulsifier agents, preferably nonionic oil-in-water emulsifier agents, preferably in quantities from about 0.1 wt % to about 20.0 wt %, based on the total agent. Quantities from about 1.0 wt % to about 10.0 wt % are particularly preferred.

It is preferred to use a powdered composition of the embodiments (A-I) to (F-I):

(A-I):

A powdered composition containing at least one particulate amorphous metal oxide in the form of a precipitated silicic acid
having a BET surface area from about 50 to about 1000 m²/g, preferably from about 100 to about 750 m²/g, particularly preferably from about 150 to about 500 m²/g (measured in each case per ISO 5794-1), and
   with the provision that
at least one waxy ester and
at least one nonionic oil-in-water emulsifier agent
   are present in a manner sorbed in said metal oxide.

(B-I):

A powdered composition containing at least one particulate amorphous metal oxide in the form of a precipitated silicic acid
having a DBP number from about 50 to about 800 g, preferably from about 100 to about 500 g, particularly preferably from about 200 to about 400 g, very particularly preferably from about 250 to about 350 g, in each case per 100 g of metal oxide, and
   with the provision that
at least one waxy ester and
at least one nonionic oil-in-water emulsifier agent
   are present in a manner sorbed in said metal oxide.

(C-I):

A powdered composition containing at least one particulate amorphous metal oxide in the form of a precipitated silicic acid
having a BET surface area from about 50 to about 1000 m²/g, preferably from about 100 to about 750 m²/g, particularly preferably from about 150 to about 500 m²/g (measured in each case per ISO 5794-1), and
having a DBP number from about 50 to about 800 g, preferably from about 100 to about 500 g, particularly preferably from about 200 to about 400 g, very particularly preferably from about 250 to about 350 g, in each case per 100 g of metal oxide,
   with the provision that
at least one waxy ester and
at least one nonionic oil-in-water emulsifier agent
   are present in a manner sorbed in said metal oxide.

(D-I):

A powdered composition containing at least one particulate amorphous metal oxide in the form of a precipitated silicic acid
having a BET surface area from about 50 to about 1000 m²/g, preferably from about 100 to about 750 m²/g, particularly preferably from about 150 to about 500 m²/g (measured in each case per ISO 5794-1),
which produces, in a 5-wt % slurry in distilled water, a pH of the resulting mixture from about 4.5 to about 8.0, in particular from about 5.0 to about 7.5,
   with the provision that
at least one waxy ester and
at least one nonionic oil-in-water emulsifier agent
   are present in a manner sorbed in said metal oxide.

(E-I):

A powdered composition containing at least one particulate amorphous metal oxide in the form of a precipitated silicic acid
having a DBP number from about 50 to about 800 g, preferably from about 100 to about 500 g, particularly preferably from about 200 to about 400 g, very particularly preferably from about 250 to about 350 g, in each case per 100 g of metal oxide, and
which produces, in a 5-wt % slurry in distilled water, a pH of the resulting mixture from about 4.5 to about 8.0, in particular from about 5.0 to about 7.5,
   with the provision that
at least one waxy ester and
at least one nonionic oil-in-water emulsifier agent
   are present in a manner sorbed in said metal oxide.

(F-I):

A powdered composition containing at least one particulate amorphous metal oxide in the form of a precipitated silicic acid
having a BET surface area from about 50 to about 1000 m²/g, preferably from about 100 to about 750 m²/g, particularly preferably from about 150 to about 500 m²/g (measured in each case per ISO 5794-1),
having a DBP number from about 50 to about 800 g, preferably from about 100 to about 500 g, particularly preferably from about 200 to about 400 g, very particularly preferably from about 250 to about 350 g, in each case per 100 g of metal oxide, and
which produces, in a 5-wt % slurry in distilled water, a pH of the resulting mixture from about 4.5 to about 8.0, in particular from about 5.0 to about 7.5,
   with the provision that
at least one waxy ester and
at least one nonionic oil-in-water emulsifier agent
   are present in a manner sorbed in said metal oxide.

The above-described waxy esters and/or the above-described utilization quantities are preferably suitable for use in the preferred embodiments (A-I) to (F-I).

Emulsifier agents suitable for use herein are selected from at least one compound of the group that is constituted from ethoxylated C₈ to C₂₄ alkanols having an average of about 8 to about 100 mol ethylene oxide per mol, ethoxylated C₈ to C₂₄ carboxylic acids having an average of about 8 to about 100 mol ethylene oxide per mol, glycerol mono- and diesters of linear saturated and unsaturated C₁₂ to C₃₀ carboxylic acids ethoxylated with an average of about 20 to about 100 mol ethylene oxide per mol, which can be hydroxylated, in particular those of myristic acid, palmitic acid, stearic acid, 12-hydroxystearic acid, or of mixtures of said fatty acids, sorbitan monoesters of linear saturated and unsaturated C₁₂ to C₃₀ carboxylic acids ethoxylated with an average of about 20 to about 100 mol ethylene oxide per mol, which can be hydroxylated, in particular those of myristic acid, palmitic acid, stearic acid, 12-hydroxystearic acid, or of mixtures of said fatty acids, silicone copolyols having ethylene oxide units or having ethylene oxide units and propylene oxide units, alkyl mono- and oligoglycosides having 8 to 22 carbon atoms in the alkyl residue and ethoxylated analogs thereof, ethoxylated sterols, partial esters of polyglycerols having n=2 to 10 glycerol units and esterified with 1 to 4 saturated or unsaturated, linear or branched, optionally hydroxylated C₈ to C₃₀ fatty acid residues, provided the latter have an HLB value of more than 7.

The ethoxylated C₈ to C₂₄ alkanols have the formula R¹O(CH₂CH₂O)ₙH, wherein R¹ denotes a linear or branched alkyl and/or alkenyl residue having 8 to 24 carbon atoms, and n (the average number of ethylene oxide units per molecule) denotes numbers from about 8 to about 100, preferably about 8 to about 30 mol ethylene oxide per 1 mol capryl alcohol, 2-ethylhexyl alcohol, capric alcohol, lauryl alcohol, isotridecyl alcohol, tridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, and brassidyl alcohol, as well as technical mixtures thereof. Adducts of about 8 to about 100 mol ethylene oxide with technical fatty alcohols having 12 to 18 carbon atoms, for example coconut, palm, palm kernel, or tallow alcohol, are also suitable.

The ethoxylated C₈ to C₂₄ carboxylic acids have the formula R¹O(CH₂CH₂O)ₙH, wherein R¹O denotes a linear or branched, saturated or unsaturated acyl residue having 8 to 24 carbon atoms, and n (the average number of ethylene oxide units per molecule) denotes numbers from about 8 to about 100, preferably about 10 to about 30 mol ethylene oxide per 1 mol caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselinic acid, arachidic acid, gadoleic acid, behenic acid, erucic acid, and brassidic acid, as well as technical mixtures thereof. Adducts of about 10 to about 100 mol ethylene oxide with technical fatty acids having 12 to 18 carbon atoms, for example coconut, palm, palm kernel, or tallow fatty acid, are also suitable. PEG-50 monostearate, PEG-100 monostearate, PEG-50 monooleate, PEG-100 monooleate, PEG-50 monolaurate, and PEG-100 monolaurate are particularly preferred.

It is particularly preferred to use C₁₂ to C₁₈ alkanols or C₁₂ to C₁₈ carboxylic acids respectively having 8 to 30 units of ethylene oxide per molecule, as well as mixtures of said substances, in particular Laureth-8, Laureth-10, Laureth-12, Laureth-20, Trideceth-8, Trideceth-9, Trideceth-10, Tri-Deceth-12, Trideceth-20, Ceteth-10, Ceteth-12, Ceteth-20, Ceteth-30, Steareth-10, Steareth-12, Steareth-20, Steareth-30, Ceteareth-10, Ceteareth-12, Ceteareth-20, Ceteareth-30, Laureth-12, and Beheneth-20.

Preferred glycerol mono- and/or diesters, ethoxylated with an average of about 20 to about 100 mol ethylene oxide per mol, of linear saturated and unsaturated C₁₂ to C₃₀ carboxylic acids, which can be hydroxylated, are selected from PEG-20 Hydrogenated Castor Oil, PEG-40 Hydrogenated Castor Oil, and PEG-60 Hydrogenated Castor Oil.

Preferred sorbitan monoesters, ethoxylated with an average of about 20 to about 100 mol ethylene oxide per mol, of linear saturated and unsaturated C₁₂ to C₃₀ carboxylic acids, which can be hydroxylated, are selected from Polysorbate-20, Polysorbate-40, Polysorbate-60, and Polysorbate-80.

C₈ to C₂₂ alkyl mono- and oligoglycosides are also preferably used. C₈ to C₂₂ alkyl mono- and oligoglycosides represent known, commercially usual emulsifier agents. They are manufactured, in particular, by reacting glucose or oligosaccharides with primary alcohols having 8 to 22 carbon atoms. In terms of the glycoside residue, both monoglycosides, in which a cyclic sugar residue is glycosidically bound to the fatty alcohol, and oligomeric glycosides having a degree of oligomerization of up to approximately 8, preferably 1 to 2, are suitable. The degree of oligomerization is a statistical average that is based on a homolog distribution that is usual for technical products of this kind. Products that are obtainable under the Plantacare® trademark contain a glucosidically bound C₈ to C₁₆ alkyl group on each oligoglucoside residue whose average degree of oligomerization is 1 to 2, in particular 1.2 to 1.4. Particularly preferred C₈ to C₂₂ alkyl mono- and oligoglycosides are selected from octyl glucoside, decyl glucoside, lauryl glucoside, palmityl glucoside, isostearyl glucoside, stearyl glucoside, arachidyl glucoside, and behenyl glucoside, as well as mixtures thereof. The acyl glucamides derived from glucamine are also suitable as nonionic oil-in-water emulsifiers.

Ethoxylated sterols, in particular ethoxylated soy sterols, also represent oil-in-water emulsifiers suitable for use herein. The degree of ethoxylation must be greater than about 5, preferably less than about 10, in order to exhibit an HLB value greater than about 7. Suitable commercial products are, for example, PEG-10 Soy Sterol, PEG-16 Soy Sterol, and PEG-25 Soy Sterol.

It is further preferred to use partial esters of polyglycerols having 2 to 10 glycerol units and esterified with 1 to 4 saturated or unsaturated, linear or branched, optionally hydroxylated C₈ to C₃₀ fatty acid esters, provided they have an HLB value of more than about 7. Diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate, and decaglycerol trihydroxystearate are particularly preferred.

Those powdered compositions are preferred which are suitable for the reshaping of keratin-containing fibers, in particular human hair, and comprise as said sorbed cosmetic active agent at least one active agent that is selected from waxy esters, setting polymers, film-forming polymers, mixtures thereof. The technical teaching of the embodiment having at least one sorbed setting polymer is combined, in the context of a preferred embodiment, with the technical teaching of the sorbed waxy esters. Combined sorption or separate sorption is possible with regard to the active agents.

At least one setting polymer is suitable as a sorbed cosmetic active agent in particular for furnishing a powdered composition for the deformation of keratin-containing fibers. Corresponding setting polymers are preferably selected from nonionic setting polymers, anionic setting polymers, amphoteric setting polymers, cationic setting polymers.

Hair-setting polymers contribute to the hold, and/or to buildup of the hair volume and hair fullness, of the overall hairstyle. These polymers are at the same time also film-forming polymers and are therefore generally typical substances for shape-imparting hair treatment agents such as hair setting agents, hair foams, hair waxes, hair sprays. It is certainly possible for film formation to be localized, and for only a few fibers to be connected to one another.

The so-called “curl retention” test is often used as a test method for the hair-setting effect of a polymer.

Film formation is included among the preferred properties of film-forming polymers. Film-forming polymers do not need to be setting polymers. “Film-forming polymers” are to be understood as those polymers which, upon drying, leave behind a continuous film on the skin, hair, or nails. Film-formers of this kind can be used in a very wide variety of cosmetic products, for example face masks, make-up, hair setting agents, hair sprays, hair gels, hair waxes, hair therapies, shampoos, or nail polishes. Those polymers which possess sufficient solubility in water or water/alcohol mixtures to be present in completely dissolved form in the powdered composition contemplated herein are particularly preferred. The film-forming polymers can be of synthetic or natural origin.

“Film-forming polymers” are furthermore understood herein as those polymers which, when used in an about 0.01 to about 20 wt % aqueous, alcoholic, or aqueous alcoholic solution, are capable of depositing a transparent polymer film onto the hair.

In the context of a preferred embodiment, the composition contains at least one nonionic setting polymer that is present in a manner sorbed in the particulate amorphous metal oxide.

A “nonionic polymer” is understood herein as a polymer that, in a protic solvent under standard conditions, carries no structural units having permanently cationic groups or anionic groups that must be respectively compensated for with counter ions in order to maintain electroneutrality. “Cationic groups” include, for example, quaternized ammonium groups but not protonated amines. “Anionic groups” include, for example, carboxyl groups and sulfonic-acid groups.

Nonionic setting polymers are contained in the powdered composition contemplated herein preferably in a quantity from about 0.01 to about 20 wt %, in particular from about 0.5 wt % to about 10 wt %, very particularly preferably from about 1.0 wt % to about 5.0 wt %, based in each case on the weight of the powdered composition.

The nonionic setting polymers are in turn preferably selected from at least one polymer of the group that is constituted from
homopolymers and nonionic copolymers of N-vinylpyrrolidone,
nonionic copolymers of isobutene,
nonionic copolymers of maleic acid anhydride.

Powdered compositions that contain, as a nonionic setting polymer, at least one polymer selected from the group that is constituted from
polyvinylpyrrolidone,
copolymers of N-vinylpyrrolidone and vinyl esters of carboxylic acids having 2 to 18 carbon atoms, in particular of N-vinylpyrrolidone and vinyl acetate,
copolymers of N-vinylpyrrolidone and N-vinylimidazole and methacrylamide,
copolymers of N-vinylpyrrolidone and N-vinylimidazole and acrylamide,
copolymers of N-vinylpyrrolidone with N,N-di-(C₁ to C₄) alkylamino-(C₂ to C₄) alkylacrylamide
   are particularly preferred.

Powdered compositions that contain, as a nonionic setting polymer, at least one polymer selected from the group that is constituted from
polyvinylpyrrolidone,
copolymers of N-vinylpyrrolidone and vinyl esters of carboxylic acids having 2 to 18 carbon atoms, in particular of N-vinylpyrrolidone and vinyl acetate,
   or mixtures of said polymers, are very particularly preferred.

Suitable polyvinylpyrrolidones are, for example, commercial products such as Luviskol® K 90 or Luviskol® K 85 of the BASF SE company.

If copolymers of N-vinylpyrrolidone and vinyl acetate are employed, it is in turn preferred if the molar ratio of the structural units of the polymer contained from the N-vinylpyrrolidone monomer to the structural units of the polymer contained from the vinyl acetate monomer is in the range of from about 20 to about 80 to about 80 to about 20, in particular from about 30 to about 70 to about 60 to about 40.

Suitable copolymerizates of vinylpyrrolidone and vinyl acetate are obtainable, for example, from the BASF SE company under the trademarks Luviskol® VA 37, Luviskol® VA 55, Luviskol® VA 64, and Luviskol® VA 73.

If what is employed as a nonionic setting polymer is at least one copolymer containing at least one structural unit in accordance with formula (M-I) and at least one structural unit in accordance with formula (M-II) and at least one structural unit in accordance with formula (M-III),

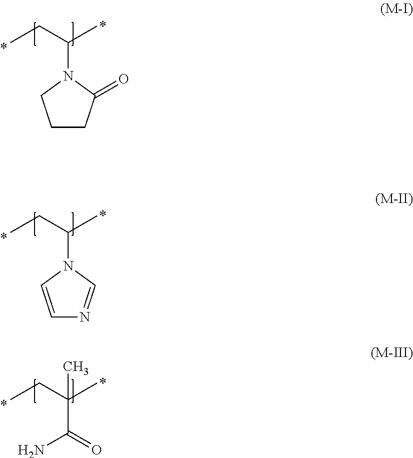

it is particularly preferred if these copolymers contain, besides polymer units that result from incorporation of the aforesaid structural units according to formulas (M-I), (M-II), and (M-III) into the copolymer, a maximum of about 5 wt %, preferably a maximum of about 1 wt %, polymer units that are based on the incorporation of other monomers. The copolymers are preferably constructed exclusively from structural units of formulas (M-I), (M-II), and (M-III) and can be described by the general formula (Poly1)

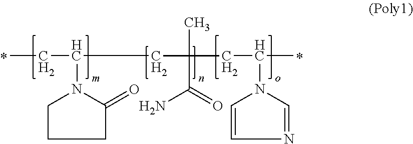

where the indices m, n, o, and p each vary depending on the molar mass of the polymer and are not intended to signify that these are block copolymers. Structural units of formulas (M-I), (M-II), and (M-III) can instead be present in statistically distributed fashion in the molecule.

A particularly preferred polymer is selected in this context from the polymers having the INCI name VP/Methacrylamide/Vinyl Imidazole Copolymer, which are obtainable e.g. under the trade name Luviset Clear from the BASF SE company.

Also suitable for use herein are those powdered compositions which contain at least one sorbed nonionic setting polymer comprising at least one structural unit of formula (M-I) and at least one structural unit of formula (M-IV)

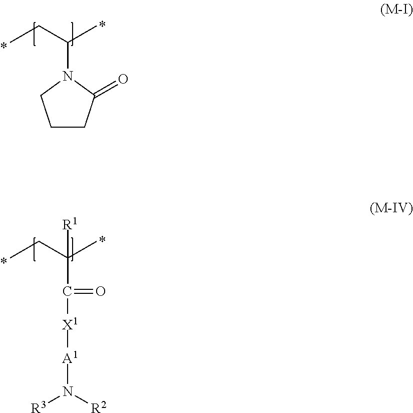

in which
R¹ denotes a hydrogen atom or a methyl group,
X¹ denotes an oxygen atom or an NH group,
A¹ denotes an ethane-1,2-diyl, propane-1,3-diyl, or butane-1,4-diyl group,
R² and R³ mutually independently denote a (C₁ to C₄) alkyl group.

It is particularly preferred if the above nonionic setting polymer is selected from at least one polymer that, for formula (M-IV), conforms to at least one or more of the following features:
R¹ signifies a methyl group,
X¹ denotes an NH group,
A¹ denotes ethane-1,2-diyl or propane-1,3-diyl,
R² and R³ mutually independently denote methyl or ethyl (particularly preferably methyl).

The group of the polymers
vinylcaprolactam/vinylpyrrolidone/dimethylaminoethyl methacrylate copolymer (for example, INCI name: Vinyl Caprolactam/PVP/Dimethylaminoethyl Methacrylate Copolymer, under the trade name Gaffix® VC 713 (ISP)),
vinylpyrrolidone/vinylcaprolactam/dimethylaminopropyl methacrylamide copolymer (e.g. INCI name: VP/Vinyl Caprolactam/DMAPA Acrylates Copolymer, under the trade name Aquaflex® SF 40 (ISP)),
vinylcaprolactam/vinylpyrrolidone/dimethylaminoethyl methacrylate copolymer (for example, as 35 to 39% solids in ethanol in the form of the commercial product Advantage LC E having the INCI name: Vinyl Caprolactam/VP/Dimethylaminoethyl Methacrylate Copolymer, Alcohol, Lauryl Pyrrolidone (ISP)),
vinylpyrrolidone/dimethylaminopropylmethacrylamide copolymer (for example, INCI name: VP/DMAPA Acrylates Copolymer, under the trade name Styleze CC-10 (10 wt % active substance) (ISP)),
   is in turn considered a preferred list for selection therefrom of at least one or more polymers.

It is preferred in the context of all the nonionic setting polymers recited (in particular those mentioned as preferred) to use one of the preferred particulate amorphous metal oxides (in particular one of the embodiments (A) to (F)) (see above) for sorption of the nonionic setting polymers.

In the context of a further preferred embodiment, the compositions contemplated herein contain at least one anionic setting polymer as a sorbed setting polymer.

Anionic setting polymers are contained in the powdered composition preferably in a quantity from about 0.1 wt % to about 20.0 wt %, particularly preferably from about 0.2 wt % to about 15.0 wt %, very particularly preferably from about 0.5 wt % to about 10.0 wt %, based in each case on the weight of the composition.

It is preferred if the anionic setting polymer contains at least one structural unit of formula (S1) that is selected from at least one structural unit of formulas (S1-1) to (S1-3)

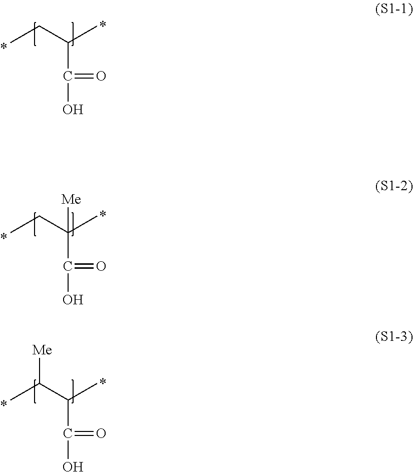

and, besides at least one structural unit of formulas (S1-1) to (S1-3), additionally contains at least one structural unit of formula (S2) that is selected from at least one structural unit of formulas (S2-1) to (S2-8)

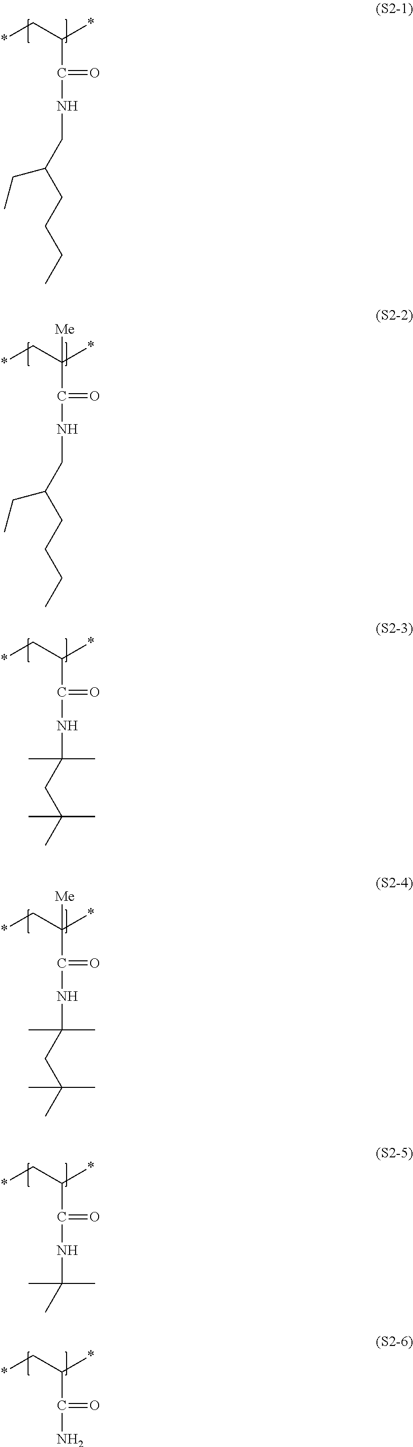

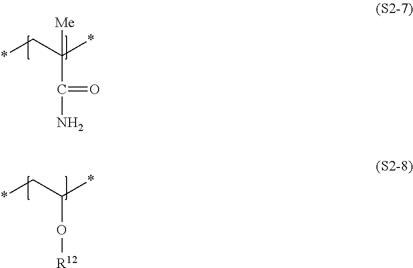

in which
R¹² denotes a (C₂ to C₁₂) acyl group (in particular acetyl or neodecanoyl).

In an exemplary embodiment, those compositions contemplated herein which contain as a sorbed setting polymer at least one polymer that contains at least one structural unit of formula (S1-3) and at least one structural unit of formula (S2-8)

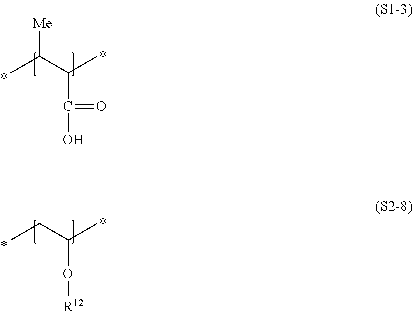

in which R¹² denotes a (C₂ to C₁₂) acyl group (in particular acetyl and/or neodecanoyl), are considered preferred.

Particularly preferred polymers of this kind are selected from at least one polymer of the group that is constituted from
copolymers of vinyl acetate and crotonic acid,
copolymers of vinyl propionate and crotonic acid,
copolymers of vinyl neodecanoate, vinyl acetate, and crotonic acid.

Such copolymers are made available, for example, by the Clariant company under the commercial name Aristoflex A 60 (INCI name: VA/Crotonates Copolymer) in an isopropanol/water mixture (60 wt % active substance), by the BASF company under the commercial name Luviset CA 66 (vinyl acetate/crotonic acid copolymer 90:10, INCI name: VA/Crotonates Copolymer), and by the National Starch company under the commercial name Resyn 28-2942 or Resyn 28-2930 (INCI name: VA/Crotonates/Vinyl Neodecanoate Copolymer).

In another embodiment, those compositions which contain as a sorbed anionic setting polymer at least one polymer that contains at least one structural unit of formula (S1-1) and at least one structural unit of formula (S2-5)

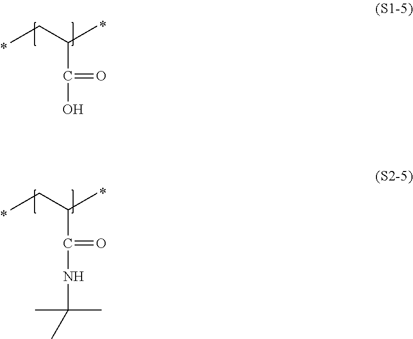

are considered preferred.

It is particularly preferred in turn if the sorbed anionic setting polymer contains, besides the above structural units of formulas (S1-1) and (S2-5), additionally at least one structural unit of formula (S3)

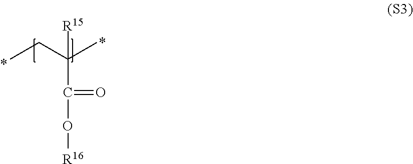

in which
R¹⁵ denotes a hydrogen atom or a methyl group,
R¹⁶ denotes a (C₁ to C₄) alkyl group (in particular a methyl group or an ethyl group).

Particularly preferred polymers of this type are selected from at least one polymer of the group that is constituted from copolymers of acrylic acid and ethyl acrylate and N-tert-butylacrylamide. Such copolymers are furnished, for example, by the BASF company under the commercial name Ultrahold® Strong (INCI name: Acrylates/t-Butylacrylamide Copolymer; a white, pourable granulate) or Ultrahold® 8 (INCI name: Acrylates/t-Butylacrylamide Copolymer, a white, pourable granulate).

It is preferred, in the context of all anionic setting polymers recited (in particular those mentioned as preferred), to use one of the preferred particulate amorphous metal oxides (in particular one of the embodiments (A) to (F)) (see above) for sorption of the anionic setting polymer.

In the context of a further preferred embodiment, the compositions contain a sorbed amphoteric setting polymer.

An “amphoteric polymer” is understood as used herein as a polymer that, in a protic solvent under standard conditions, carries structural units having anionic groups that must be compensated for with counter ions in order to maintain electroneutrality, and additionally comprises structural units having groups cationizable by protonation but is free of permanently cationized groups. “Anionic” groups include carboxyl groups and sulfonic-acid groups. “Permanently cationized” nitrogen atoms are to be understood as those nitrogen atoms which carry a positive charge and thereby form a quaternary ammonium compound. By definition, N-oxide-containing polymers are also included among the amphoteric polymers.

Amphoteric setting polymers are contained in the powdered composition contemplated herein preferably in a quantity from about 0.1 wt % to about 20.0 wt %, particularly preferably from about 0.2 wt % to about 15.0 wt %, very particularly preferably from about 0.5 wt % to about 10.0 wt %, based in each case on the weight of the composition.

It is suitable if the amphoteric setting polymer contains at least one structural unit of formula (S1) that is selected from at least one structural unit of formulas (S1-1) to (S1-3)

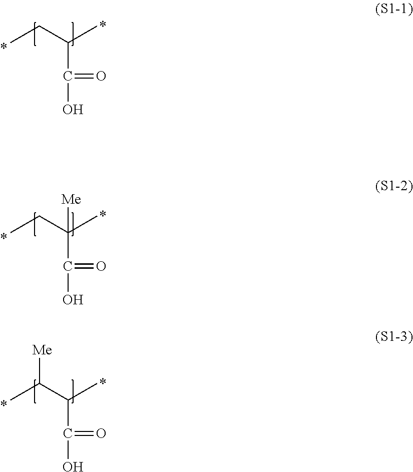

and besides at least one structural unit of formulas (S1-1) to (S1-3) additionally contains at least one structural unit of formula (S2) that is selected from at least one structural unit of formulas (S2-9) to (S2-15)

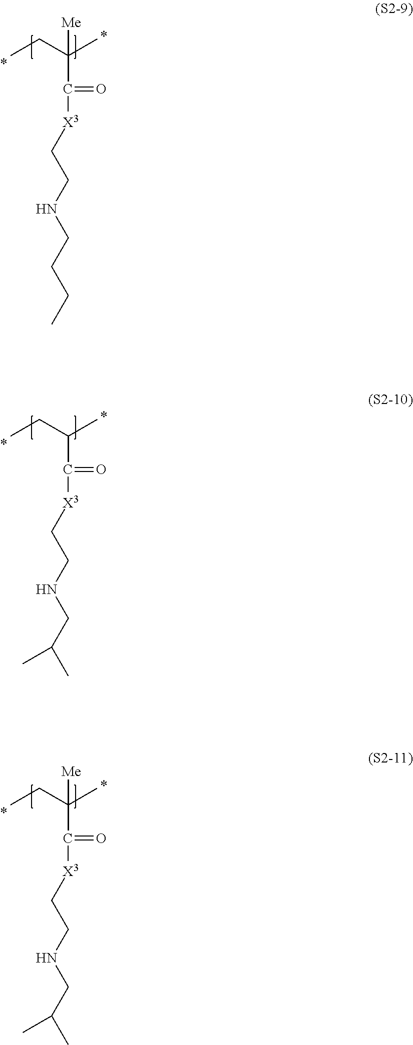

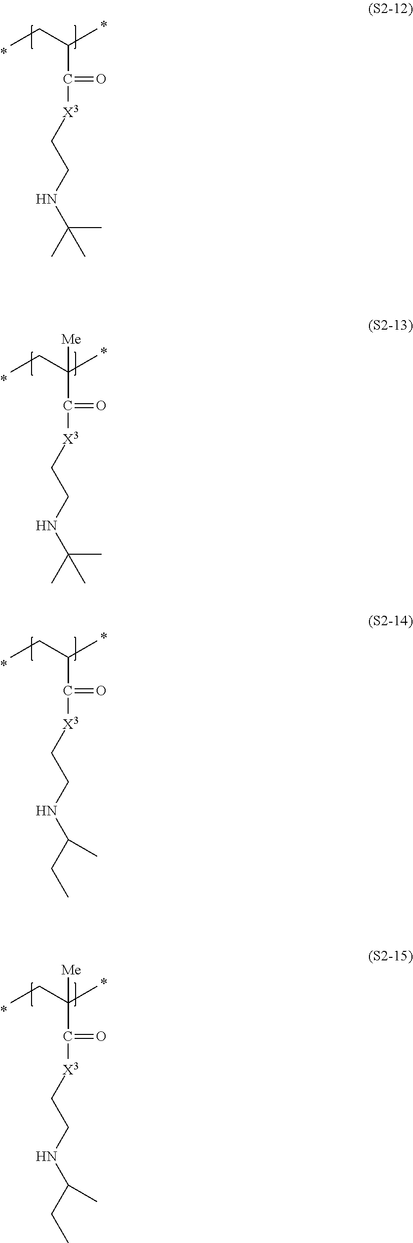

in which
X³ denotes an oxygen atom or an NH group.

It is in turn suitable if the amphoteric setting polymer additionally comprises, besides at least one structural unit of formulas (S1-1) to (S1-3) and at least one structural unit of formulas (S2-9) to (S2-15), at least one structural unit of formulas (S2-1) to (S2-8)

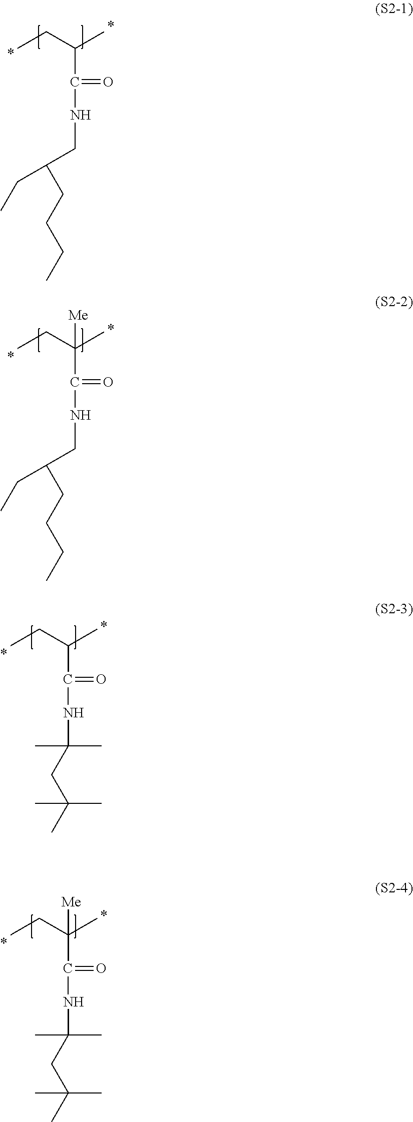

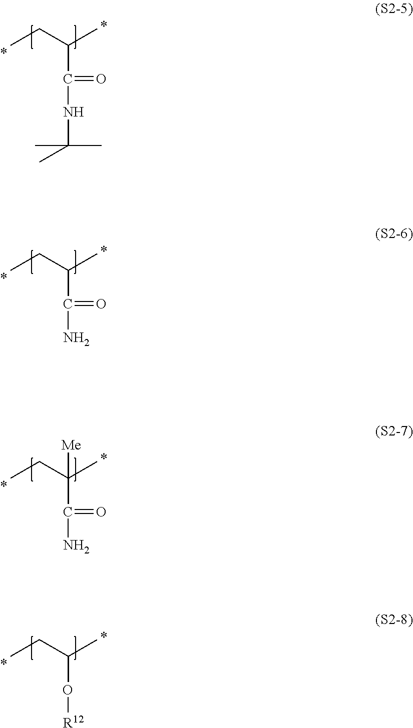

in which
R¹² denotes a (C₂ to C₁₂) acyl group (in particular acetyl or neodecanoyl).

An amphoteric setting polymer that contains at least one structural unit of formula (S1-1), at least one structural unit of formula (S2-3), and at least one structural unit of formula (S2-16) (selected in particular from the group that is constituted from the above formulas (S2-5) to (S2-12) with the provision that X³ denotes an oxygen atom)

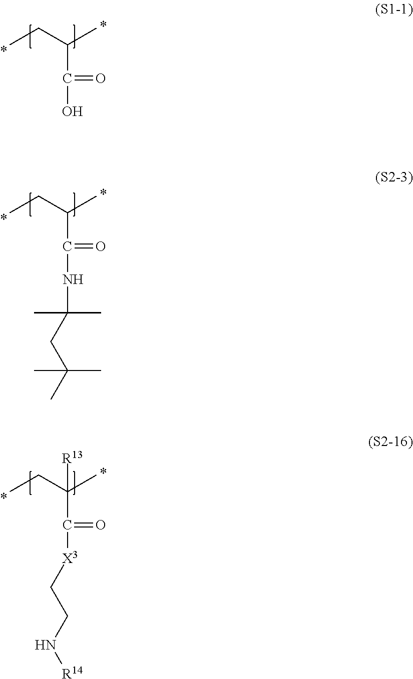

in which X³ denotes an oxygen atom or an NH group,
R¹³ denotes a hydrogen atom or a methyl group, and
R¹⁴ denotes an alkyl group having 4 carbon atoms (in particular n-butyl, sec-butyl, isobutyl, or tert-butyl),
is preferably suitable.

It is particularly suitable in turn if the amphoteric setting polymer additionally contains, besides the above structural units of formulas (S1-1), (S2-3), and (S2-16), at least one structural unit of formula (S3)

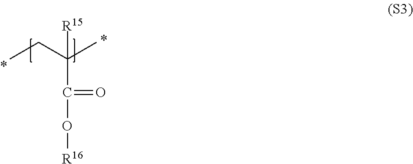

in which
R¹⁵ denotes a hydrogen atom or a methyl group,
R¹⁶ denotes a (C₁ to C₄) alkyl group (in particular a methyl group or an ethyl group).

Preferred polymers of this kind are selected from the group that is constituted from copolymers of acrylic acid, (C₁ to C₄) alkyl acrylate, N—(C₄ alkyl)aminoethyl methacrylate, and N—(C₈ alkyl)acrylamide.

An example of an amphoteric setting polymer particularly preferably usable in the context of this embodiment is the polymer obtainable under the commercial name Amphomer® from the National Starch company, having the INCI name Octylacrylamide/Acrylates/Butylaminoethyl Methacrylate Copolymer.

A further particularly preferred sorbed amphoteric setting polymer comprises
at least one monomer A1 selected from acrylic acid, methacrylic acid, acrylic acid alkyl esters, and methacrylic acid alkyl esters, and
at least one amphoteric monomer A2 selected from (meth)acryloylalkylamine oxides of formula A2-I

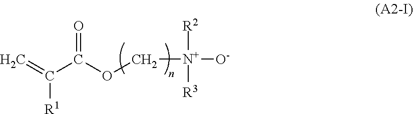

or (meth)acryloylalkyl betaines of formula A2-II

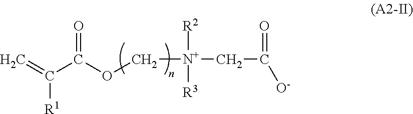

wherein in formula A2-I and formula A2-II
R¹ denotes H or CH₃,
R² and R³ mutually independently each denote optionally branched C₁₋₁₀ alkyl, and
n denotes an integer from 1 to 20.

For purposes herein, what are to be understood as “amphoteric setting polymers” that are constituted from the aforesaid monomers are only those copolymers which contain, besides polymer units that result from incorporation of the aforesaid monomers A1 and A2 into the copolymer, a maximum of about 5 wt %, preferably a maximum of about 1 wt % of polymer units that are attributable to the incorporation of other monomers. Copolymers A preferably are constructed exclusively from polymer units that result from incorporation of the aforesaid monomers A1 and A2 into the copolymer.

Preferred monomers A1 are acrylic acid, methacrylic acid, acrylic acid C₁₋₂₀ alkyl esters, and methacrylic acid C₁₋₂₀ alkyl esters.

Particularly preferably, monomer A1 is selected from acrylic acid, methacrylic acid, acrylic acid methyl ester, methacrylic acid methyl ester, acrylic acid ethyl ester, methacrylic acid ethyl ester, acrylic acid propyl ester, methacrylic acid propyl ester, acrylic acid isopropyl ester, methacrylic acid isopropyl ester, acrylic acid lauryl ester, methacrylic acid lauryl ester, acrylic acid cetyl ester, methacrylic acid cetyl ester, acrylic acid stearyl ester, and methacrylic acid stearyl ester, very particularly preferably from acrylic acid, methacrylic acid, acrylic acid methyl ester, methacrylic acid methyl ester, acrylic acid ethyl ester, methacrylic acid ethyl ester, acrylic acid lauryl ester, methacrylic acid lauryl ester, acrylic acid stearyl ester, and methacrylic acid stearyl ester.

Preferred monomers A2 are (meth)acryloylalkylamine oxides of formula A2-I and/or (meth)acryloylalkyl betaines of formula A2-II, wherein R² and R³ denote, mutually independently in each case, methyl, ethyl, propyl, isopropyl, butyl, isobutyl, or tert-butyl, particularly preferably methyl.

Preferred monomers A2 are furthermore selected from at least one monomer from the group that is constituted from (meth)acryloylalkylamine oxides of formula A2-I and/or (meth)acryloylalkyl betaines of formula A2-II, wherein n respectively denotes an integer from 1 to 5, preferably an integer from 1 to 3, and particularly preferably denotes 2.

Monomers A2 as well are preferably selected from at least one monomer from the group that is constituted from (meth)acryloylalkylamine oxides of formula A2-I and/or (meth)acryloylalkyl betaines of formula A2-II, wherein R¹ respectively denotes CH₃.

Particularly preferably, monomers A2 are selected from at least one monomer from the group that is constituted from (meth)acryloylalkylamine oxides of formula A2-I and/or (meth)acryloylalkyl betaines of formula A2-II, wherein R² and R³ mutually independently denote in each case methyl, ethyl, propyl, isopropyl, butyl, isobutyl, or tert-butyl, particularly preferably methyl, n denotes in each case an integer from 1 to 5, preferably an integer from 1 to 3 and particularly preferably 2, and R¹ respectively denotes CH₃.

Very particularly preferably, monomer A2 is selected from at least one monomer from the group that is constituted from (meth)acryloylalkylamine oxides of formula A2 and/or (meth)acryloylalkyl betaines of formula A2-II, wherein R¹, R², and R³ respectively denote CH₃ and n denotes 2.

It is furthermore preferred, for all the embodiments previously discussed, that said amphoteric setting polymer be constituted (in particular, exclusively) from at least one monomer of formula (A1) and at least one monomer, corresponding to the respective embodiment, of formula (A2-I).

In a particularly preferred embodiment the agent contemplated herein contains at least one amphoteric setting polymer that is constituted from
at least two monomers A1, wherein the first monomer is selected from acrylic acid, methacrylic acid, acrylic acid methyl ester, methacrylic acid methyl ester, acrylic acid ethyl ester, methacrylic acid ethyl ester, acrylic acid propyl ester, methacrylic acid propyl ester, acrylic acid isopropyl ester, and methacrylic acid isopropyl ester, and the second monomer is selected from acrylic acid stearyl ester and methacrylic acid stearyl ester, and
as monomer A2, methacryloylethylamine oxide, in particular methacryloylethyl-N,N-dimethylamine oxide (in formula (A2-I): R¹=CH₃, n=2, R² and R³=CH₃).

These copolymers, too, are known and are obtainable, for example, under the name Diaformer Z-632 from the Clariant company, the use of Diaformer Z-632 being particularly preferred.

In a preferred embodiment, the agent contains at least one amphoteric setting polymer that is constituted from
at least three monomers A1, wherein the first monomer is selected from acrylic acid, methacrylic acid, acrylic acid methyl ester, methacrylic acid methyl ester, acrylic acid ethyl ester, methacrylic acid ethyl ester, acrylic acid propyl ester, methacrylic acid propyl ester, acrylic acid isopropyl ester, and methacrylic acid isopropyl ester, the second monomer is selected from acrylic acid lauryl ester and methacrylic acid lauryl ester, and the third monomer is selected from acrylic acid stearyl ester and methacrylic acid stearyl ester and
as monomer A2, methacryloylethylamine oxide in particular methacryloylethyl-N,N-dimethylamine oxide (in formula (A2-I): R¹=CH₃, n=2, R² and R³=CH₃).

Corresponding copolymers are likewise known and are obtainable e.g. under the designations Diaformer Z-611, Diaformer Z-612, Diaformer Z-613, Diaformer Z-631, Diaformer Z-633, Diaformer Z-651, Diaformer Z-711N, Diaformer Z-712N, and Diaformer Z-731N from the Clariant company; the use of Diaformer Z-712N and Diaformer Z-651 is preferred.

It is preferred, in the context of all amphoteric setting polymers recited (in particular those mentioned as preferred), to use one of the preferred particulate amorphous metal oxides (in particular one of the embodiments (A) to (F)) (see above) for sorption of the amphoteric setting polymer.

At least one film-forming polymer is suitable as a sorbed cosmetic active agent, in particular for furnishing a powdered composition for the deformation of keratinic fibers. Corresponding film-forming polymers are preferably selected from cationic film-forming polymers or/and from silicones. Among them are, in part, polymers that are also setting polymers.

“Cationic” polymers are to be understood as polymers that comprise in the main chain and/or side chain a group that is “permanently” cationic. For purposes herein, those polymers that comprise a cationic group regardless of the pH of the agent are referred to as “permanently cationic.” These are, as a rule, polymers that contain a quaternary nitrogen atom, for example in the form of an ammonium group. Preferred cationic groups are quaternary ammonium groups. Those polymers in particular in which the quaternary ammonium group is bonded via a C₁₋₄ hydrocarbon group to a main polymer chain constructed from acrylic acid, methacrylic acid, or derivatives thereof, have proven particularly suitable.

A cationic film-forming polymer that is preferably suitable for use herein is at least one cationic film-forming polymer that contains at least one structural element of formula (M9) and additionally at least one structural element of formula (M10)

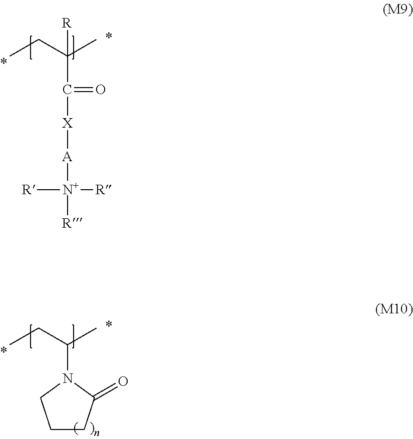

in which
R denotes a hydrogen atom or a methyl group,
R′, R″, and R″ mutually independently denote a (C₁ to C₃₀) alkyl group,
X denotes an oxygen atom or an NH group,
A denotes an ethane-1,2-diyl group or a propane-1,3-diyl group,
n signifies 1 or 3.

All possible physiologically acceptable anions, for example chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge.

Also suitable are those cationic film-forming polymers which comprise at least one structural unit of formula (M5), at least one structural unit of formula (V), and at least one structural unit of formula (VI),

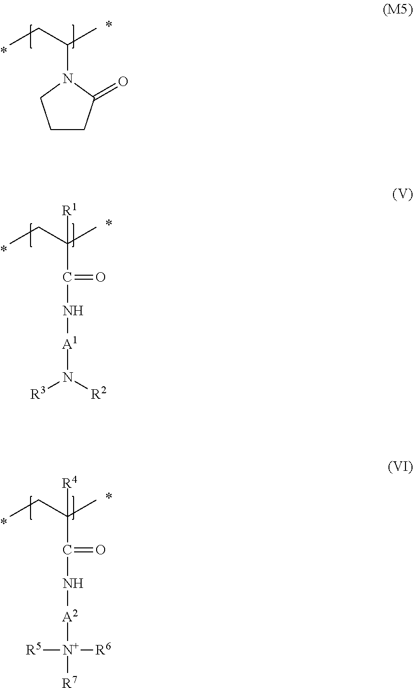

in which
R¹ and R⁴ mutually independently denote a hydrogen atom or a methyl group,
A¹ and A² mutually independently denote an ethane-1,2-diyl, propane-1,3-diyl, or butane-1,4-diyl group,
R², R³, R⁵, and R⁶ mutually independently denote a (C₁ to C₄) alkyl group,
R⁷ denotes a (C₈ to C₃₀) alkyl group.

All possible physiologically acceptable anions, for example chloride, bromide, hydrogen sulfate, methyl sulfate, ethyl sulfate, tetrafluoroborate, phosphate, hydrogen phosphate, dihydrogen phosphate or p-toluenesulfonate, triflate, serve to compensate for the positive polymer charge of monomer (VI).

Suitable compounds are commercialized, for example, as
copolymers of dimethylaminoethyl methacrylate, quaternized with diethyl sulfate, with N-vinylpyrrolidone, having the INCI name Polyquaternium-11, under the designations Gafquat® 440, Gafquat® 734, Gafquat® 755 (each ISP company) and Luviquat PQ 11 PN (BASF SE),
copolymers of N-vinylpyrrolidone, N-(3-dimethylaminopropyl) methacrylamide), and 3-(methacryloylamino)propyllauryldimethylammonium chloride (INCI name Polyquaternium-55), which are marketed e.g. under the commercial names Styleze W-10 or Styleze W 20 (10 or 20 wt % active substance in ethanol-water mixture) by the ISP company,
copolymers of N-vinylpyrrolidone, N-vinylcaprolactam, N-(3-dimethylaminopropyl) methacrylamide), and 3-(methacryloylamino)propyllauryldimethylammonium chloride (INCI name Polyquaternium-69), which are marketed e.g. under the commercial name AquaStyle® 300 (28 to 32 wt % active substance in ethanol-water mixture) by the ISP company.

Powdered compositions (K1) that contain at least one particulate amorphous metal oxide in which at least one cationic film-forming polymer, which contains at least one structural element of formula (M9) and additionally at least one structural element of formula (M10)

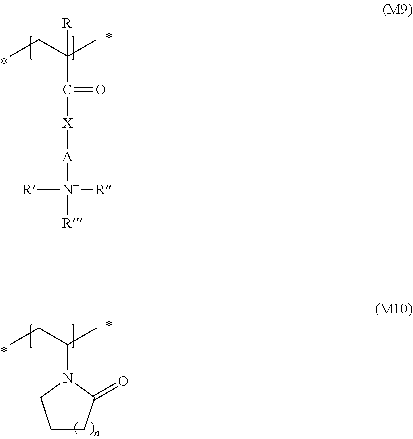

in which
R denotes a hydrogen atom or a methyl group,
R′, R″, and R″ mutually independently denote a (C₁ to C₃₀) alkyl group,
X denotes an oxygen atom or an NH group,
A denotes an ethane-1,2-diyl group or a propane-1,3-diyl group,
n signifies 1 or 3
is present in sorbed fashion, are thus in particular considered very particularly preferred.

The preferred particulate amorphous metal oxides (see above) are furthermore regarded, mutatis mutandis, as preferred therefor.

The sorbed cationic film-forming polymers are furthermore selected particularly preferably from cationic quaternized cellulose derivatives.

Those cationic quaternized celluloses which carry in a side chain more than one permanent cationic charge prove to be particularly advantageous for purposes herein. Among these cationic celluloses, those cationic celluloses having the INCI name Polyquaternium-4, which are marketed e.g. under the designations Celquat® H 100, Celquat® L 200 by the National Starch company, are in turn particularly suitable.

Those powdered compositions (K2) containing at least one particulate amorphous metal oxide in which Polyquaternium-4 is present in sorbed fashion as a cationic film-forming polymer, are thus considered very particularly preferred. The preferred particulate amorphous metal oxides (see above) are regarded, mutatis mutandis, as preferred therefor.

Those cationic film-forming polymers which comprise at least one structural element of formula (M11)

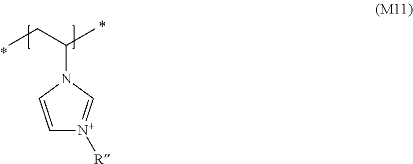

in which R″ denotes a (C₁ to C₄) alkyl group, in particular a methyl group, and additionally at least one further cationic and/or nonionic structural element, serve as cationic polymers that are particularly preferably usable for purposes herein.

All possible physiologically acceptable anions, for example chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge.

It is in turn preferred if at least one copolymer (co1), which comprises besides at least one structural element of formula (M11) additionally a structural element of formula (M5)

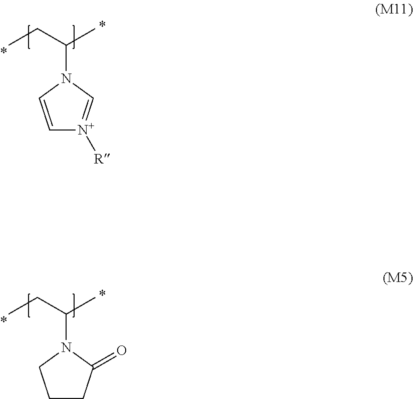

in which R″ denotes a (C₁ to C₄) alkyl group, in particular a methyl group,
is sorbed as a cationic film-forming polymer.

All possible physiologically acceptable anions, for example chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge of copolymers (co1).

Very particularly preferred cationic film-forming and/or cationic setting polymers as copolymers (co1) contain about 10 to about 30 mol %, preferably about 15 to about 25 mol %, and in particular about 20 mol % structural units in accordance with formula (M11) and about 70 to about 90 mol %, preferably about 75 to about 85 mol %, and in particular about 80 mol % structural units in accordance with formula (M5).

It is particularly preferred in this context if copolymers (co1) contain, besides polymer units that result from incorporation of the aforesaid structural units in accordance with formulas (M11) and (M5) into the copolymer, a maximum of about 5 wt %, preferably a maximum of about 1 wt %, polymer units that are based on the incorporation of other monomers. Copolymers (co1) are preferably constructed exclusively from structural units of formula (M11), where R″=methyl, and (M5).

If a chloride ion is used to compensate for the positive charge of the copolymer, these N-methylvinylimidazole/vinylpyrrolidone copolymers are then referred to according to INCI nomenclature as Polyquaternium-16 and are obtainable e.g. from BASF under the commercial names Luviquat® Style, Luviquat® FC 370, Luviquat® FC 550, Luviquat® FC 905, and Luviquat® HM 552.

If a methosulfate is used to compensate for the positive charge of the copolymer, these N-methylvinylimidazole/vinylpyrrolidone copolymers are then referred to according to INCI nomenclature as Polyquaternium-44 and are obtainable e.g. from BASF under the commercial names Luviquat® UltraCare.

Those powdered compositions (K3) containing at least one particulate amorphous metal oxide in which at least one cationic film-forming polymer that comprises besides at least one structural element of formula (M11) additionally a structural element of formula (M5)

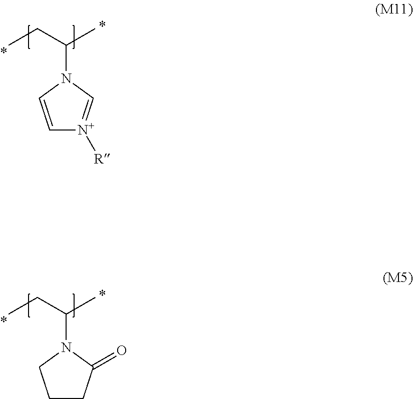

in which R″ denotes a (C₁ to C₄) alkyl group, in particular a methyl group,
is present in sorbed fashion, are thus considered very particularly preferred.

The preferred particulate amorphous metal oxides (see above) are regarded, mutatis mutandis, as preferred therefor.

In addition to or instead of the copolymer or copolymers (co1), the agents contemplated herein can also contain copolymers (co2) that, proceeding from copolymer (co1), contain as additional structural units those of formula (M6)

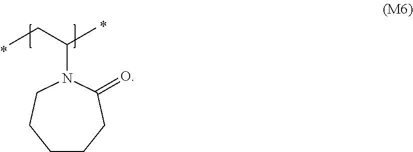

Further particularly preferred agents are thus characterized in that they contain as a cationic film-forming polymer at least one copolymer (co2) that contains at least one structural unit in accordance with formula (M11-a) and at least one structural unit in accordance with formula (M5) and at least one structural unit in accordance with formula (M6).

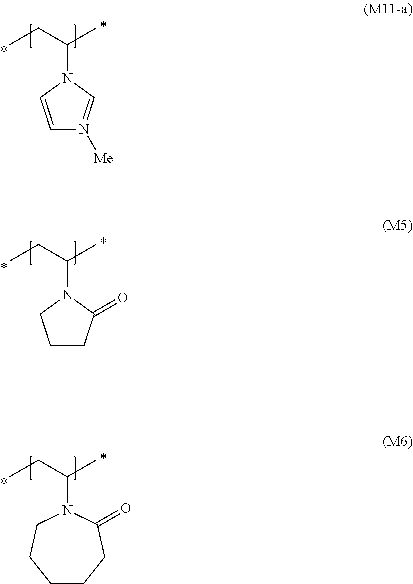

Here as well, it is particularly preferred if copolymers (co2) contain, besides polymer units that result from the incorporation of the aforesaid structural units in accordance with formulas (M11-a), (M5), and (M6) into the copolymer, a maximum of about 5 wt %, preferably a maximum of about 1 wt %, polymer units that are based on the incorporation of other monomers. Copolymers (co2) are preferably constructed exclusively from structural units of formulas (M11-a), (M5), and (M6).

All possible physiologically acceptable anions, for example chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge of the (co2) component.

If a methosulfate is used to compensate for the positive charge of the copolymer, these N-methylvinylimidazole/vinylpyrrolidone/vinylcaprolactam copolymers are then referred to according to INCI nomenclature as Polyquaternium-46 and are obtainable e.g. from BASF under the commercial name Luviquat® Hold.

Very particularly preferred copolymers (co2) contain about 1 to about 20 mol %, preferably about 5 to about 15 mol %, and in particular about 10 mol % structural units in accordance with formula (M11-a) and about 30 to about 50 mol %, preferably about 35 to about 45 mol %, and in particular about 40 mol % structural units in accordance with formula (M5) and about 40 to about 60 mol %, preferably about 45 to about 55 mol %, and in particular about 60 mol % structural units in accordance with formula (M6).

In addition to or instead of the copolymer or copolymers (co1) and/or (co2), the agents herein can also contain as a cationic film-forming polymer copolymers (co3) that comprise, as structural units, structural units of formulas (M-11a) and (M5) as well as further structural units from the group of vinylimidazole units and further structural units from the group of acrylamide unit and/or methacrylamide units.

Further particularly preferred agents are characterized in that they contain, as a sorbed cationic film-forming polymer, at least one copolymer (co3) that contains at least one structural unit in accordance with formula (M11-a) and at least one structural unit in accordance with formula (M5) and at least one structural unit in accordance with formula (M10) and at least one structural unit in accordance with formula (M12)

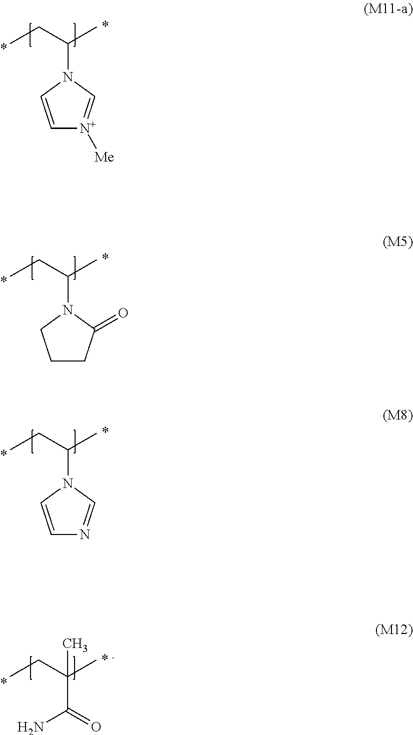

Here as well, it is particularly preferred if copolymers (co3) contain, besides polymer units that result from incorporation of the aforesaid structural units in accordance with formulas (M11-a), (M5), (M8), and (M12) into the copolymer, a maximum of about 5 wt %, preferably a maximum of about 1 wt %, polymer units that are based on the incorporation of other monomers. Copolymers (co3) are preferably constructed exclusively from structural units of formulas (M11-a), (M5), (M8), and (M12).

All possible physiologically acceptable anions, for example chloride, bromide, hydrogen sulfate, methyl sulfate, ethyl sulfate, tetrafluoroborate, phosphate, hydrogen phosphate, dihydrogen phosphate, or p-toluenesulfonate, triflate, serve to compensate for the positive polymer charge of the (co3) component.

If a methosulfate is used to compensate for the positive charge of the copolymer, these N-methylvinylimidazole/vinylpyrrolidone/vinylimidazole/methacrylamide copolymers are referred to according to INCI nomenclature as Polyquaternium-68 and are obtainable e.g. from BASF under the commercial name Luviquat® Supreme.

Very particularly preferred copolymers (co3) contain about 1 to about 12 mol %, preferably about 3 to about 9 mol %, and in particular about 6 mol % structural units in accordance with formula (M11-a) and about 45 to about 65 mol %, preferably about 50 to about 60 mol %, and in particular about 55 mol % structural units in accordance with formula (M5) and about 1 to about 20 mol %, preferably about 5 to about 15 mol %, and in particular about 10 mol % structural units in accordance with formula (M8) and about 20 to about 40 mol %, preferably about 25 to about 35 mol %, and in particular about 29 mol % structural units in accordance with formula (M12).

Among the film-forming cationic polymers selected from the cationic polymers having at least one structural element of the above formula (M11-a), those considered preferred are:
vinylpyrrolidone/1-vinyl-3-methyl-1H-imidazolium chloride copolymers (for example the one having the INCI name Polyquaternium-16, under the commercial designations Luviquat® Style, Luviquat® FC 370, Luviquat® FC 550, Luviquat® FC 905, and Luviquat® HM 552 (BASF SE)),
vinylpyrrolidone/1-vinyl-3-methyl-1H-imidazolium methyl sulfate copolymers (for example the one having the INCI name Polyquaternium-44, under the commercial designations Luviquat® Care (BASF SE)),
vinylpyrrolidone/vinylcaprolactam/1-vinyl-3-methyl-1H-imidazolium terpolymers (for example the one having the INCI name Polyquaternium-46, under the commercial designations Luviquat® Care or Luviquat® Hold (BASF SE)),
vinylpyrrolidone/methacrylamide/vinylimidazole/1-vinyl-3-methyl-1H-imidazolium methyl sulfate copolymers (for example the one having the INCI name Polyquaternium-68, under the commercial designation Luviquat® Supreme (BASF SE)),
   as well as mixtures of said polymers.

It is preferred in the context of all the cationic film-forming polymers recited (in particular those mentioned as preferred) to use one of the preferred particulate amorphous metal oxides (in particular one of the embodiments (A) to (F)) (see above), in particular in the preferred quantities or quantity ratios, for sorption of the cationic film-forming polymers.

Utilization of at least one silicone is particularly favorable for decreasing or avoiding particle deposition, visible to the naked eye, of the particulate metal oxide on the keratin-containing fibers.

At least one silicone oil and/or at least one silicone gum is preferably used as a silicone.

Silicone oils or silicone gums suitable for use herein are, in particular, dialkyl- and alkylarylsiloxanes, for example dimethylpolysiloxane and methylphenylpolysiloxane, as well as alkoxylated, quaternized, or also anionic derivatives thereof. Cyclic and linear polydialkylsiloxanes, alkoxylated and/or aminated derivatives thereof, dihydroxypolydimethylsiloxanes, and polyphenylalkylsiloxanes are preferred.

Silicone oils produce a wide variety of effects. For example, they simultaneously influence dry and wet combability, the feel of the dry and wet hair, and shine. The skilled artisan understands the term “silicone oils” as several structures of organosilicon compounds. It is understood firstly as dimethiconols.

The following commercial products are recited as examples of such products: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401DC (all the aforesaid Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all the aforesaid Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both Guardian Laboratories), Nonychosine E, Nonychosine V (both Exsymol), SanSurf Petrolatum-25, Satin Finish (both Collaborative Laboratories, Inc.), Silatex-D30 (Cosmetic Ingredient Resources), Silsoft 148, Silsoft E-50, Silsoft E-623 (all the aforesaid Crompton Corporation), SM555, SM2725, SM2765, SM2785 (all the aforesaid GE Silicones), Taylor T-SiI CD-1, Taylor TME-4050E (all Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all the aforesaid Wacker-Chemie GmbH).

Dimethicones constitute the second group of silicones that can be contained. They can be both linear and branched, and also cyclic or cyclic and branched.

Dimethicone copolyols constitute a further group of silicones that are suitable. Corresponding dimethicone copolyols are commercially obtainable and are marketed, for example, by the Dow Corning company under the designation Dow Corning® 5330 Fluid.

The teaching herein also, of course, encompasses the fact that dimethiconols, dimethicones, and/or dimethicone copolymers can already be present as an emulsion. The corresponding emulsion of dimethiconols, dimethicones, and/or dimethicone copolyols can be produced both after manufacture of the corresponding dimethiconols, dimethicones, and/or dimethicone copolyols, from them and using usual emulsification methods known to one skilled in the art. For this purpose both cationic, anionic, nonionic, or zwitterionic surfactants and emulsifier agents can be used, as auxiliaries, as adjuvants for manufacture of the corresponding emulsions. The emulsions of dimethiconols, dimethicones, and/or dimethicone copolyols can of course also be manufactured directly by way of an emulsion polymerization method. Such methods, too, are very familiar to one skilled in the art.

If dimethiconols, dimethicones, and/or dimethicone copolyols are used as an emulsion, the droplet size of the emulsified particles is then, according to an embodiment, equal to about 0.01 to about 10,000 μm, preferably about 0.01 to about 100 μm, particularly preferably about 0.01 to about 20 μm, and very particularly preferably about 0.01 to about 10 μm. The particle size is determined using the light-scattering method.

If branched dimethiconols, dimethicones, and/or dimethicone copolyols are used, this is to be understood to mean that the branching is greater than a random branching that occurs randomly as a result of impurities in the respective monomers. “Branched” dimethiconols, dimethicones, and/or dimethicone copolyols are therefore to be understood, for purposes herein, to mean that the degree of branching is greater than about 0.01%. A degree of branching greater than about 0.1% is preferred, and very particularly preferably it is greater than about 0.5%. The degree of branching is determined from the ratio of unbranched monomers to the branching monomers, i.e. to the quantity of tri- and tetrafunctional siloxanes. Both low-branching and high-branching dimethiconols, dimethicones, and/or dimethicone copolyols can be very particularly preferred.

Particularly suitable silicones are aminofunctional silicones, in particular the silicones grouped under the INCI name Amodimethicone. It is therefore preferred if the agents contemplated herein additionally contain at least one aminofunctional silicone. These are to be understood as silicones that comprise at least one optionally substituted amino group. These silicones are referred to according to the INCI declaration as Amodimethicone, and are obtainable, for example, in the form of an emulsion as a commercial product Dow Corning® 939, or as a commercial product Dow Corning® 949, mixed with a cationic and a nonionic surfactant.

Those aminofunctional silicones which have an amine number above about 0.25 meq/g, preferably above about 0.3 meq/g, and particularly preferably above about 0.4 meq/g are preferably used. The amine number here denotes the milliequivalent of amine per gram of the aminofunctional silicone; it can be ascertained by titration, and can also be indicated with the “mg KOH/g” unit.

Quaternized aminosilicones are likewise preferably suitable. A particularly preferred quaternized aminosilicone carries the INCI name Quaternium-80 and is marketed by the Evonik Goldschmidt company under the commercial designation Abil Quat®.

The agents contain the silicones preferably in quantities from about 0.01 wt % to about 30 wt %, particularly preferably from about 0.05 to about 10.0 wt %, based on the total agent.

It is preferred in the context of all the silicones recited (in particular those mentioned as preferred) to use one of the preferred particulate amorphous metal oxides (in particular one of the embodiments (A) to (F)) (see above), in particular in the preferred quantities or quantity ratios, for sorption of the silicone.

In an exemplary embodiment, at least one oxidation dye precursor is suitable as a cosmetic active agent sorbed, in particular in order to furnish a powdered composition for the coloring of keratin-containing fibers. Corresponding oxidation dye precursors are preferably selected from at least one developer component and at least one coupler component.

One skilled in the art is familiar with so-called “developer components” and “coupler components” as oxidation dye precursors. The developer components, under the influence of oxidizing agents or atmospheric oxygen, form the actual dyes with one another or by coupling to one or more coupler components. Oxidizing coloring agents are notable for outstanding, long-lasting color results.

In an embodiment, a p-phenylenediamine derivative or a physiologically acceptable salt thereof is used as a developer component. Particularly preferred are p-phenylenediamine derivatives of formula (E1)

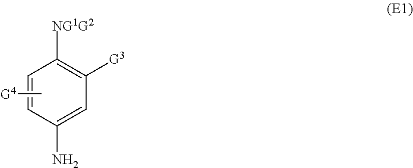

wherein
G¹ denotes a hydrogen atom, a (C₁ to C₄) alkyl residue, a (C₁ to C₄) monohydroxyalkyl residue, a (C₂ to C₄) polyhydroxyalkyl residue, a (C₁ to C₄) alkoxy-(C₁ to C₄) alkyl residue, a 4′-aminophenyl residue, or a (C₁ to C₄) alkyl residue that is substituted with a nitrogen-containing group, with a phenyl residue, or with a 4′-aminophenyl residue;
G² denotes a hydrogen atom, a (C₁ to C₄) alkyl residue, a (C₁ to C₄) monohydroxyalkyl residue, a (C₂ to C₄) polyhydroxyalkyl residue, a (C₁ to C₄) alkoxy-(C₁ to C₄) alkyl residue or a (C₁ to C₄) alkyl residue that is substituted with a nitrogen-containing group;
G³ denotes a hydrogen atom, a halogen atom such as a chlorine, bromine, iodine, or fluorine atom, a (C₁ to C₄) alkyl residue, a (C₁ to C₄) monohydroxyalkyl residue, a (C₂ to C₄) polyhydroxyalkyl residue, a (C₁ to C₄) hydroxyalkoxy residue, a (C₁ to C₄) alkoxy-(C₁ to C₄) alkyl residue, a (C₁ to C₄) acetylaminoalkoxy residue, a mesylamino-(C₁ to C₄) alkoxy residue, or a (C₁ to C₄) carbamoylaminoalkoxy residue;
G⁴ denotes a hydrogen atom, a halogen atom, or a (C₁ to C₄) alkyl residue, or a (C₁ to C₄) alkoxy-(C₁ to C₄) alkyl residue; or
if G³ are G⁴ are in the ortho-position with respect to one another, they can together form a bridging α,ω-alkylenedioxo group, for example an ethylenedioxy group.

Particularly preferred p-phenylenediamines of formula (E1) are selected from one or more compounds of the group that is constituted from p-phenylenediamine, p-toluylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)aniline, N,N-bis-(β-hydroxyethyl)-p-phenylenediamine, 4-N,N-bis-(β-hydroxyethyl)amino-2-methylaniline, 4-N,N-bis-(β-hydroxyethyl)amino-2-chloroaniline, 2-(β-hydroxyethyl)-p-phenylenediamine, 2-(α,β-dihydroxyethyl)-p-phenylenediamine, 2-fluoro-p-phenylenediamine, 2-isopropyl-p-phenylenediamine, N-(β-hydroxypropyl)-p-phenylenediamine, 2-hydroxymethyl-p-phenylenediamine, N,N-dimethyl-3-methyl-p-phenylenediamine, N,N-(ethyl,β-hydroxyethyl)-p-phenylenediamine, N-(β,γ-dihydroxypropyl)-p-phenylenediamine, N-(4′-aminophenyl)-p-phenylenediamine, N-phenyl-p-phenylenediamine, 2-(β-hydroxyethyloxy)-p-phenylenediamine, 2-methoxymethyl-p-phenylenediamine, 2-(β-acetylaminoethyloxy)-p-phenylenediamine, N-(β-methoxyethyl)-p-phenylenediamine, N-(4-amino-3-methylphenyl)-N-[3-(1H-imidazole-1-yl)propyl]amine, and 5,8-diaminobenzo-1,4-dioxane, as well as physiologically acceptable salts thereof

p-Phenylenediamine derivatives of formula (E1) that are very particularly preferred are selected from at least one compound of the group: p-phenylenediamine, p-toluylenediamine, 2-(β-hydroxyethyl)-p-phenylenediamine, 2-(α,β-dihydroxyethyl)-p-phenylenediamine, N,N-bis-(β-hydroxyethyl)-p-phenylenediamine, 2-methoxymethyl-p-phenylenediamine, N-(4-amino-3-methylphenyl)-N-(3-(1H-imidazol-1-yl)propylamine, and physiologically acceptable salts of said compounds.

In another embodiment, compounds that contain at least two aromatic nuclei that are substituted with amino groups and/or hydroxyl groups are used as developer components.

Among the binuclear developer components that can be used in the coloring compositions herein may be cited, in particular, those compounds which correspond to formula (E2) below, as well as physiologically acceptable salts thereof:

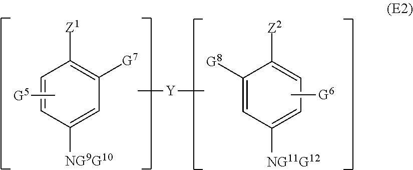

wherein
Z¹ and Z² mutually independently denote a hydroxyl residue or NH₂ residue that is optionally substituted with a (C₁ to C₄) alkyl residue, with a (C₁ to C₄) hydroxyalkyl residue, and/or with a bridge Y, or that optionally is part of a bridging ring system;
the bridge Y denotes an alkylene group having 1 to 14 carbon atoms, for example a linear or branched alkylene chain or an alkylene ring, which can be interrupted or terminated by one or more nitrogen-containing groups and/or by one or more heteroatoms such as oxygen, sulfur, or nitrogen atoms, and possibly can be substituted with one or more hydroxyl or (C₁ to C₈) alkoxy residues, or a direct bond;
G⁵ and G⁶ mutually independently denote a hydrogen atom or halogen atom, a (C₁ to C₄) alkyl residue, a (C₁ to C₄) monohydroxyalkyl residue, a (C₂ to C₄) polyhydroxyalkyl residue, a (C₁ to C₄) aminoalkyl residue, or a direct bond to the bridge Y,
G⁷, G⁸, G⁹, G¹⁰, G¹¹ and G¹² mutually independently denote a hydrogen atom, a direct bond to the bridge Y, or a (C₁ to C₄) alkyl residue,
   with the provision that the compounds of formula (E2) contain only one bridge Y per molecule.

The substituents used in formula (E2) are defined analogously to the statements made above.

Preferred binuclear developer components of formula (E2) are selected in particular from at least one of the following compounds: N,N′-bis-(β-hydroxyethyl)-N,N′-bis-(4′-aminophenyl)-1,3-diaminopropan-2-ol, N,N′-bis-(β-hydroxyethyl)-N,N′-bis-(4′-aminophenyl)ethylenediamine, N,N′-bis-(4-aminophenyl)tetramethylenediamine, N,N′-bis-(β-hydroxyethyl)-N,N′-bis-(4-aminophenyl)tetramethylenediamine, N,N′-bis-(4′-(methylamino)phenyl)tetramethylenediamine, N,N′-diethyl-N,N′-bis-(4′-amino-3′-methylphenyl)ethylenediamine, bis-(2-hydroxy-5-aminophenyl)methane, N,N′-bis-(4′-aminophenyl)-1,4-diazacycloheptane, N,N′-bis-(2-hydroxy-5-aminobenzyl)piperazine, N-(4′-aminophenyl)-p-phenylenediamine, and 1,10-bis-(2′,5′-diaminophenyl)-1,4,7,10-tetraoxadecane, and physiologically acceptable salts thereof.

Very particularly preferred binuclear developer components of formula (E2) are selected from N,N′-bis-(β-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, and 1,10-bis-(2,5-diaminophenyl)-1,4,7,10-tetraoxadecane, or a physiologically acceptable salt of said compounds.

In yet another embodiment, at least one p-aminophenol derivative or a physiologically acceptable salt thereof is used as a developer component. p-Aminophenol derivatives of formula (E3) are particularly preferred:

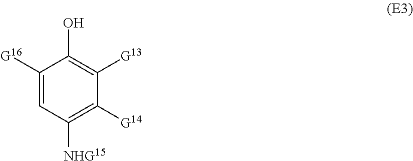

wherein
G¹³ denotes a hydrogen atom, a halogen atom, a (C₁ to C₄) alkyl residue, a (C₁ to C₄) monohydroxyalkyl residue, a (C₂ to C₄) polyhydroxyalkyl residue, a (C₁ to C₄) alkoxy-(C₁ to C₄) alkyl residue, a (C₁ to C₄) aminoalkyl residue, a hydroxy-(C₁ to C₄) alkylamino residue, a (C₁ to C₄) hydroxyalkoxy residue, a (C₁ to C₄ hydroxyalkyl-(C₁ to C₄) aminoalkyl residue, or a (di-[(C₁ to C₄)alkyl]amino)-(C₁ to C₄) alkyl residue, and
G¹⁴ denotes a hydrogen atom or halogen atom, a (C₁ to C₄) alkyl residue, a (C₁ to C₄) monohydroxyalkyl residue, a (C₂ to C₄) polyhydroxyalkyl residue, a (C₁ to C₄) alkoxy-(C₁ to C₄) alkyl residue, a (C₁ to C₄) aminoalkyl residue, or a (C₁ to C₄) cyanoalkyl residue,
G¹⁵ denotes hydrogen, a (C₁ to C₄) alkyl residue, a (C₁ to C₄) monohydroxyalkyl residue, a (C₂ to C₄) polyhydroxyalkyl residue, a phenyl residue, or a benzyl residue, and
G¹⁶ denotes hydrogen or a halogen atom.

The substituents used in formula (E3) are defined analogously to the statements above.

Preferred p-aminophenols of formula (E3) are, in particular, p-aminophenol, N-methyl-p-aminophenol, 4-amino-3-methylphenol, 4-amino-3-fluorophenol, 2-hydroxymethylamino-4-aminophenol, 4-amino-3-hydroxymethylphenol, 4-amino-2-(β-hydroxyethoxy)phenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-(α,β-dihydroxyethyl)phenol, 4-amino-2-fluorophenol, 4-amino-2-chlorophenol, 4-amino-2,6-dichlorophenol, 4-amino-2-(diethylaminomethyl)phenol, and physiologically acceptable salts thereof

Very particularly preferred compounds of formula (E3) are p-aminophenol, 4-amino-3-methylphenol, 4-amino-2-aminomethylphenol, 4-amino-2-(α,β-dihydroxyethyl)phenol, and 4-amino-2-(diethylaminomethyl)phenol.

The developer component can furthermore be selected from o-aminophenol and derivatives thereof, for example 2-amino-4-methylphenol, 2-amino-5-methylphenol, or 2-amino-4-chlorophenol.

The developer component can moreover be selected from heterocyclic developer components, for example from pyrimidine derivatives, pyrazole derivatives, pyrazolopyrimidine derivatives, or physiologically acceptable salts thereof

Preferred pyrimidine derivatives are selected from compounds according to formula (E4) and physiologically acceptable salts thereof:

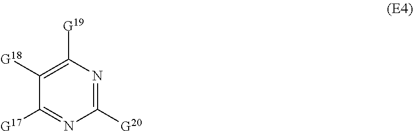

in which
G¹⁷, G¹⁸, and G¹⁹ mutually independently denote a hydrogen atom, a hydroxy group, a (C₁ to C₄) alkoxy group, or an amino group; and
G²⁰ denotes a hydroxy group or an —NG²¹G²² group in which G²¹ and G²² mutually independently denote a hydrogen atom, a (C₁ to C₄) alkyl group, a (C₁ to C₄) monohydroxyalkyl group;
   with the provision that a maximum of two of the groups from G¹⁷, G¹⁸, G¹⁹, and G²⁰ signify a hydroxy group, and at most two of the residues G¹⁷, G¹⁸, and G¹⁹ denote a hydrogen atom. It is in turn preferred if, in accordance with formula (E4), at least two groups from G¹⁷, G¹⁸, G¹⁹, and G²⁰ denote an —NG²¹G²² group, and at most two groups from G¹⁷, G¹⁸, G¹⁹, and G²⁰ denote a hydroxy group.

Particularly preferred pyrimidine derivatives are, in particular, the compounds 2,4,5,6-tetraaminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine, and 2,5,6-triaminopyrimidine.

Preferred pyrazole derivatives are selected from compounds according to formula (E5),

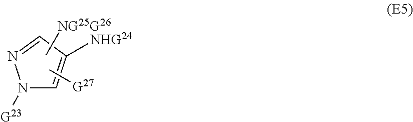

in which
G²³, G²⁴, G²⁵ mutually independently denote a hydrogen atom, a (C₁ to C₄) alkyl group, a (C₁ to C₄) monohydroxyalkyl group, a (C₂ to C₄) polyhydroxyalkyl group, an optionally substituted aryl group, or an optionally substituted aryl-(C₁ to C₄) alkyl group, with the provision that if G²⁵ denotes a hydrogen atom, G²⁷ can denote not only the aforesaid groups but additionally an —NH₂ group;
G²⁶ denotes a hydrogen atom, a (C₁ to C₄) alkyl group, a (C₁ to C₄) monohydroxyalkyl group, or a (C₂ to C₄) polyhydroxyalkyl group; and
G²⁷ denotes a hydrogen atom, an optionally substituted aryl group, a (C₁ to C₄) alkyl group, or a (C₁ to C₄) monohydroxyalkyl group, in particular a hydrogen atom or a methyl group.

In formula (E5) the —NG²⁵G²⁶ residue preferably binds to the −5 position on the pyrazole cycle, and the G²⁷ residue to the −3 position.

Particularly preferred pyrazole derivatives are in particular the compounds that are selected from 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(β-aminoethyl)amino-1,3-dimethylpyrazole, and physiologically acceptable salts thereof

Very particularly preferred developer components are selected from at least one compound from the group that is constituted from p-phenylenediamine, p-toluylenediamine, 2-(β-hydroxyethyl)-p-phenylenediamine, 2-(α,β-dihydroxyethyl)-p-phenylenediamine, N,N-bis-(β-hydroxyethyl)-p-phenylenediamine, N-(4-amino-3-methylphenyl)-N-[3-(1H-imidazol-1-yl)propyl]amine, N,N′-bis-(β-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(α,β-dihydroxyethyl)phenol and 4-amino-2-(diethylaminomethyl)phenol, 4,5-diamino-1-(β-hydroxyethyl)pyrazole, 2,4,5,6-tetraaminopyrimidine, 4-hydroxy-2,5,6-triaminopyrimidine, 2-hydroxy-4,5,6-triaminopyrimidine, and physiologically acceptable salts of said compounds.

Examples of the residues recited as substituents of the compounds of formulas (E1) to (E5) will be listed below: examples of (C₁ to C₄) alkyl residues are the groups —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃.

Examples of (C₁ to C₄) alkoxy residues are —OCH₃, —OCH₂CH₃, —OCH₂CH₂CH₃, —OCH(CH₃)₂, —OCH₂CH₂CH₂CH₃, —OCH₂CH(CH₃)₂, —OCH(CH₃)CH₂CH₃, —OC(CH₃)₃, in particular a methoxy group or ethoxy group.

In addition, preferred examples of a (C₁ to C₄) monohydroxyalkyl group that can be used are —CH₂OH, —CH₂CH₂OH, —CH₂CH₂CH₂OH, —CHCH(OH)CH₃, —CH₂CH₂CH₂CH₂OH, wherein the —CH₂CH₂OH group is preferred.

A particularly preferred example of a (C₂ to C₄) polyhydroxyalkyl group is the 1,2-dihydroxyethyl group.

Examples of halogen atoms are F, Cl, or Br atoms; Cl atoms are very particularly preferred examples.

Examples of nitrogen-containing groups are, in particular, —NH₂, (C₁ to C₄) monoalkylamino groups, (C₁ to C₄) dialkylamino groups, (C₁ to C₄) trialkylammonium groups, (C₁ to C₄) monohydroxyalkylamino groups, imidazolinium, and —NH₃⁺.

Examples of (C₁ to C₄) monoalkylamino groups are —NHCH₃, —NHCH₂CH₃, —NHCH₂CH₂CH₃, —NHCH(CH₃)₂.

Examples of a (C₁ to C₄) dialkylamino group are —N(CH₃)₂, —N(CH₂CH₃)₂.

Examples of (C₁ to C₄) trialkylammonium groups are —N⁺(CH₃)₃, —N⁺(CH₃)₂(CH₂CH₃), —N⁺(CH₃)(CH₂CH₃)₂.

Examples of (C₁ to C₄) hydroxyalkylamino residues are —NH—CH₂CH₂OH, —NH—CH₂CH₂CH₂OH.

Examples of (C₁ to C₄) alkoxy-(C₁ to C₄) alkyl groups are the —CH₂CH₂—O—CH₃, —CH₂CH₂CH₂—O—CH₃, —CH₂CH₂—O—CH₂CH₃, —CH₂CH₂CH₂—O—CH₂CH₃, —CH₂CH₂—O—CH(CH₃), —CH₂CH₂CH₂—O—CH(CH₃) groups.

Examples of hydroxy-(C₁ to C₄) alkoxy residues are —O—CH₂OH, —O—CH₂CH₂OH, —O—CH₂CH₂CH₂OH, —O—CHCH(OH)CH₃, —O—CH₂CH₂CH₂CH₂OH.

Examples of (C₁ to C₄) acetylaminoalkoxy residues are —O—CH₂NHC(O)CH₃, —O—CH₂CH₂NHC(O)CH₃, —O—CH₂CH₂CH₂NHC(O)CH₃, —O—CH₂CH(NHC(O)CH₃)CH₃, —O—CH₂CH₂CH₂CH₂NHC(O)CH₃.

Examples of (C₁ to C₄) carbamoylaminoalkoxy residues are —O—CH₂CH₂—NH—C(O)—NH₂, —O—CH₂CH₂CH₂—NH—C(O)—NH₂, —O—CH₂CH₂CH₂CH₂—NH—C(O)—NH₂.

Examples of (C₁ to C₄) aminoalkyl residues are —CH₂NH₂, —CH₂CH₂NH₂, —CH₂CH₂CH₂NH₂, —CH₂CH(NH₂)CH₃, —CH₂CH₂CH₂CH₂NH₂.

Examples of (C₁ to C₄) cyanoalkyl residues are —CH₂CN, —CH₂CH₂CN, —CH₂CH₂CH₂CN.

Examples of (C₁ to C₄) hydroxyalkylamino-(C₁ to C₄) alkyl residues are —CH₂CH₂NH—CH₂CH₂OH, —CH₂CH₂CH₂NH—CH₂CH₂OH, —CH₂CH₂NH—CH₂CH₂CH₂OH, —CH₂CH₂CH₂NH—CH₂CH₂CH₂OH.

Examples of di[(C₁ to C₄) hydroxyalkyl]amino-(C₁ to C₄) alkyl residues are —CH₂CH₂N(CH₂CH₂OH)₂, —CH₂CH₂CH₂N(CH₂CH₂OH)₂, —CH₂CH₂N(CH₂CH₂CH₂OH)₂, —CH₂CH₂CH₂N(CH₂CH₂CH₂OH)₂.

An example of aryl groups is the phenyl group.

Examples of aryl-(C₁ to C₄) alkyl groups are the benzyl group and the 2-phenylethyl group.

Coupler components alone do not produce any significant color in the context of oxidative coloring, but instead always require the presence of developer components. It is therefore preferred that when at least one developer component is used, at least one coupler component is additionally utilized.

Coupler components for use herein allow at least one chemical residue of the coupler to be substituted with the oxidized form of the developer component, in which context a covalent bond forms between the coupler component and developer component. Couplers are preferably cyclic compounds that carry on the cycle at least two groups selected from (i) optionally substituted amino groups, and/or (ii) hydroxyl groups. If the cyclic compound is a six-membered ring (preferably aromatic), the aforesaid groups are then located preferably in the ortho- or meta-position with respect to one another.

Coupler components as contemplated herein are preferably selected from at least one component of one of the following classes:
m-aminophenol and/or derivatives thereof,
m-diaminobenzene and/or derivatives thereof,
o-diaminobenzene and/or derivatives thereof,
o-aminophenol derivatives, for example o-aminophenol,
naphthalene derivatives having at least one hydroxy group,
di- or trihydroxybenzene and/or derivatives thereof,
pyridine derivatives,
pyrimidine derivatives,
monohydroxyindole derivatives and/or monoaminoindole derivatives,
monohydroxyindoline derivatives and/or monoaminoindoline derivatives
pyrazolone derivatives, for example 1-phenyl-3-methylpyrazol-5-one,
morpholine derivatives, for example 6-hydroxybenzomorpholine or 6-aminobenzomorpholine,
quinoxaline derivatives, for example 6-methyl-1,2,3,4-tetrahydroquinoxaline;
   mixtures of two or more compounds from one or more of these classes are likewise preferred in the context of this embodiment.

The m-aminophenols or derivatives thereof usable herein are preferably selected from at least one compound of formula (K1) and/or from at least one physiologically acceptable salt of a compound in accordance with formula (K1),

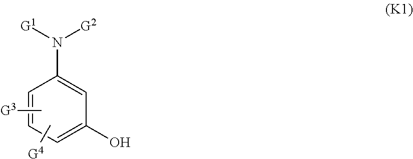

in which
G¹ and G² mutually independently denote a hydrogen atom, a (C₁ to C₄) alkyl group, a (C₃ to C₆) cycloalkyl group, a (C₂ to C₄) alkenyl group, a (C₁ to C₄) monohydroxyalkyl group, a (C₂ to C₄) polyhydroxyalkyl group, a (C₂ to C₄) perfluoracyl group, an aryl-(C₁ to C₆) alkyl group, an amino-(C₁ to C₆) alkyl group, a (C₁ to C₆) dialkylamino-(C₁ to C₆) alkyl group, or a (C₁ to C₆) alkoxy-(C₁ to C₆) alkyl group, wherein G¹ and G², together with the nitrogen atom, can form a five-, six, or seven-membered ring;
G³ and G⁴ mutually independently denote a hydrogen atom, a halogen atom, a (C₁ to C₄) alkyl group, a (C₁ to C₄) alkoxy group, a hydroxy group, a (C₁ to C₄) monohydroxyalkyl group, a (C₂ to C₄) polyhydroxyalkyl group, a hydroxy-(C₁ to C₄) alkoxy group, a (C₁ to C₆) alkoxy-(C₂ to C₆) alkoxy group, an aryl group, or a heteroaryl group.

Particularly preferred m-aminophenol coupler components are selected from at least one compound from the group that is constituted from m-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-(diethylamino)phenol, N-cyclopentyl-3-aminophenol, 1,3-dihydroxy-5-(methylamino)benzene, 3-ethylamino-4-methylphenol, 2,4-dichloro-3-aminophenol, and physiologically acceptable salts of all the compounds recited above.

The m-diaminobenzenes or derivatives thereof usable herein are preferably selected from at least one compound of formula (K2) and/or from at least one physiologically acceptable salt of a compound in accordance with formula (K2),

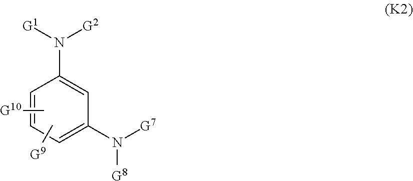

in which
G⁵, G⁶, G⁷, and G⁸ mutually independently denote a hydrogen atom, a (C₁ to C₄) alkyl group, a (C₃ to C₆) cycloalkyl group, a (C₂ to C₄) alkenyl group, a (C₁ to C₄) monohydroxyalkyl group, a (C₂ to C₄) polyhydroxyalkyl group, a (C₁ to C₄) alkoxy-(C₁ to C₄) alkyl group, an aryl-(C₁ to C₄) alkyl group, a heteroaryl-(C₁ to C₄) alkyl group, a (C₂ to C₄) perfluoracyl group, or form, together with the nitrogen atom, a five- or six-membered heterocycle;
G⁹ and G¹⁰ mutually independently denote a hydrogen atom, a halogen atom, a (C₁ to C₄) alkyl group, an ω-(2,4-diaminophenyl)-(C₁ to C₄) alkyl group, an ω-(2,4-diaminophenyloxy)-(C₁ to C₄) alkoxy group, a (C₁ to C₄) alkoxy group, a hydroxy group, a (C₁ to C₄) alkoxy-(C₂ to C₄) alkoxy group, an aryl group, a heteroaryl group, a (C₁ to C₄) monohydroxyalkyl group, a (C₂ to C₄) polyhydroxyalkyl group, a hydroxy-(C₁ to C₄) alkoxy group.

Particularly preferred m-diaminobenzene coupler components are selected from at least one compound from the group that is constituted from m-phenylenediamine, 2-(2,4-diaminophenoxy)ethanol, 1,3-bis(2,4-diaminophenoxy)propane, 1-methoxy-2-amino-4-(2′-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2′-hydroxyethyl)aminobenzene, and physiologically acceptable salts of all the compounds recited above.

The o-diaminobenzenes or derivatives thereof usable herein are preferably selected from at least one compound of formula (K3) and/or from at least one physiologically acceptable salt of a compound in accordance with formula (K3),

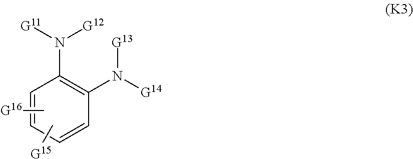

in which
G¹¹, G¹², G¹³, and G¹⁴ mutually independently denote a hydrogen atom, a (C₁ to C₄) alkyl group, a (C₃ to C₆) cycloalkyl group, a (C₂ to C₄) alkenyl group, a (C₁ to C₄) monohydroxyalkyl group, a (C₂ to C₄) polyhydroxyalkyl group, a (C₁ to C₄) alkoxy-(C₁ to C₄) alkyl group, an aryl-(C₁ to C₄) alkyl group, a heteroaryl-(C₁ to C₄) alkyl group, a (C₂ to C₄) perfluoracyl group, or form, together with the nitrogen atom, a five- or six-membered heterocycle;
G¹⁵ and G¹⁶ mutually independently denote a hydrogen atom, a halogen atom, a carboxyl group, a (C₁ to C₄) alkyl group, a (C₁ to C₄) alkoxy group, a hydroxy group, a (C₁ to C₄) monohydroxyalkyl group, a (C₂ to C₄) polyhydroxyalkyl group, a hydroxy-(C₁ to C₄) alkoxy group.

Particularly preferred o-diaminobenzene coupler components are selected from at least one compound from the group that is constituted from 3,4-diaminobenzoic acid and 2,3-diamino-1-methylbenzene, and physiologically acceptable salts of all the compounds recited above.

Preferred di- or trihydroxybenzenes and derivatives thereof are selected from at least one compound from the group that is constituted from resorcinol, resorcinol monomethyl ether, 2-methylresorcinol, 5-methylresorcinol, 2,5-dimethylresorcinol, 2-chlororesorcinol, 4-chlororesorcinol, pyrogallol, and 1,2,4-trihydroxybenzene.

The pyridine derivatives usable herein are preferably selected from at least one compound of formula (K4) and/or from at least one physiologically acceptable salt of a compound in accordance with formula (K4),

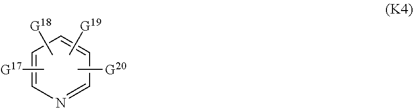

in which
G¹⁷ and G¹⁸ mutually independently denote a hydroxy group or an —NG²¹G²² group, in which G²¹ and G²² mutually independently denote a hydrogen atom, a (C₁ to C₄) alkyl group, a (C₃ to C₆) cycloalkyl group, a (C₂ to C₄) alkenyl group, an aryl group, a (C₁ to C₄) monohydroxyalkyl group, a (C₂ to C₄) polyhydroxyalkyl group, a (C₁ to C₄) alkoxy-(C₁ to C₄) alkyl group, an aryl-(C₁ to C₄) alkyl group, a heteroaryl-(C₁ to C₄) alkyl group,
G¹⁹ and G²⁰ mutually independently denote a hydrogen atom, a halogen atom, a (C₁ to C₄) alkyl group, or a (C₁ to C₄) alkoxy group.

It is preferred if, in accordance with formula (K4), the G¹⁷ and G¹⁸ residues are in the ortho- or meta-position with respect to one another.

Particularly preferred pyridine derivatives are selected from at least one compound of the group that is constituted from 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2,6-dimethoxypyridine, 3,4-diaminopyridine, 2-(2-methoxyethyl)amino-3-amino-6-methoxypyridine, 2-(4′-methoxyphenyl)amino-3-aminopyridine, and physiologically acceptable salts of the aforesaid compounds.

Preferred naphthalene derivatives having at least one hydroxy group are selected from at least one compound of the group that is constituted from 1-naphthol, 2-methyl-1-naphthol, 2-hydroxymethyl-1-naphthol, 2-hydroxyethyl-1-naphthol, 1,3-dihydroxynaphthalene, 1,5-dihydroxynaphthalene, 1,6-dihydroxynaphthalene, 1,7-dihydroxynaphthalene, 1,8-dihydroxynaphthalene, 2,7-dihydroxynaphthalene, and 2,3-dihydroxynaphthalene.

The indole derivatives usable herein are preferably selected from at least one compound of formula (K5) and/or from at least one physiologically acceptable salt of a compound in accordance with formula (K5),

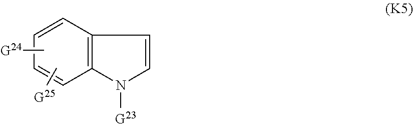

in which
G²³ denotes a hydrogen atom, a (C₁ to C₄) alkyl group, a (C₃ to C₆) cycloalkyl group, a (C₂ to C₄) alkenyl group, a (C₁ to C₄) monohydroxyalkyl group, a (C₂ to C₄) polyhydroxyalkyl group, an aryl-(C₁ to C₄) alkyl group,
G²⁴ denotes a hydroxy group or an —NG²⁶G²⁷ group, in which G²⁶ and G²⁷ mutually independently denote a hydrogen atom, a (C₁ to C₄) alkyl group, a (C₃ to C₆) cycloalkyl group, a (C₂ to C₄) alkenyl group, a (C₁ to C₄) monohydroxyalkyl group, a (C₂ to C₄) polyhydroxyalkyl group,
G²⁵ a hydrogen atom, a halogen atom, or a (C₁ to C₄) alkyl group,
   with the provision that G²⁴ binds in the meta- or ortho-position with respect to the structural fragment NG²³ of the formula.

Particularly preferred indole derivatives are selected from at least one compound of the group that is constituted from 4-hydroxyindole, 6-hydroxyindole, and 7-hydroxyindole, and physiologically acceptable salts of the aforesaid compounds.

The indoline derivatives usable herein are preferably selected from at least one compound of formula (K6) and/or from at least one physiologically acceptable salt of a compound in accordance with formula (K6),

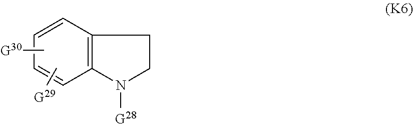

in which
G²⁸ denotes a hydrogen atom, a (C₁ to C₄) alkyl group, a (C₃ to C₆) cycloalkyl group, a (C₂ to C₄) alkenyl group, a (C₁ to C₄) monohydroxyalkyl group, a (C₂ to C₄) polyhydroxyalkyl group, an aryl-(C₁ to C₄) alkyl group,
G²⁹ denotes a hydroxy group or an —NG³¹G³² group, in which G³¹ and G³² mutually independently denote a hydrogen atom, a (C₁ to C₄) alkyl group, a (C₃ to C₆) cycloalkyl group, a (C₂ to C₄) alkenyl group, a (C₁ to C₄) monohydroxyalkyl group, a (C₂ to C₄) polyhydroxyalkyl group,
G³⁰ a hydrogen atom, a halogen atom, or a (C₁ to C₄) alkyl group,
   with the provision that G²⁹ binds in the meta- or ortho-position with respect to structural fragment NG²⁸ of the formula.

Particularly preferred indoline derivatives are selected from at least one compound of the group that is constituted from 4-hydroxyindoline, 6-hydroxyindoline, and 7-hydroxyindoline, and physiologically acceptable salts of the aforesaid compounds.

Preferred pyrimidine derivatives are selected from at least one compound of the group that is constituted from 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine, and 4,6-dihydroxy-2-methylpyrimidine, and physiologically acceptable salts of the aforesaid compounds.

Coupler components particularly preferred are selected from m-aminophenol, 5-amino-2-methylphenol, 3-amino-2-chloro-6-methylphenol, 2-hydroxy-4-aminophenoxyethanol, 5-amino-4-chloro-2-methylphenol, 5-(2′-hydroxyethyl)amino-2-methylphenol, 2,4-dichloro-3-aminophenol, o-aminophenol, m-phenylenediamine, 2-(2,4-diaminophenoxy)ethanol, 1,3-bis(2,4-diaminophenoxy)propane, 1-methoxy-2-amino-4-(2′-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2′-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of these compounds, or physiologically acceptable salts of the aforesaid compounds.

The coupler components are used preferably in a quantity from about 0.005 to about 20 wt %, preferably about 0.1 to about 5 wt %, based in each case on the corresponding powdered oxidizing coloring agent.

Developer components and coupler components are generally used in approximately molar quantities with respect to one another. Although molar use has proven useful, a certain excess of individual oxidation dye precursors is not disadvantageous, so that developer components and coupler components can be present at a molar ratio from about 1:0.5 to about 1:3, in particular about 1:1 to about 1:2.

Examples of the residues recited as substituents of the compounds of formulas (K1) to (K6) are listed below: Examples of (C₁ to C₄) alkyl residues are the groups —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃.

Examples of (C₃ to C₆) cycloalkyl groups are the cyclopropyl, cyclopentyl, and cyclohexyl group.

Examples of (C₁ to C₄) alkoxy residues are —OCH₃, —OCH₂CH₃, —OCH₂CH₂CH₃, —OCH(CH₃)₂, —OCH₂CH₂CH₂CH₃, —OCH₂CH(CH₃)₂, —OCH(CH₃)CH₂CH₃, —OC(CH₃)₃, in particular a methoxy group or ethoxy group.

Preferred examples of a (C₁ to C₄) monohydroxyalkyl group that can be recited are furthermore —CH₂OH, —CH₂CH₂OH, —CH₂CH₂CH₂OH, —CH₂CH(OH)CH₃, —CH₂CH₂CH₂CH₂OH, wherein the —CH₂CH₂OH group is preferred.

A particularly preferred example of a (C₂ to C₄) polyhydroxyalkyl group is the 1,2-dihydroxyethyl group.

Examples of halogen atoms are F, Cl, or Br atoms; Cl atoms are very particularly preferred examples.

Examples of nitrogen-containing groups are in particular —NH₂, (C₁ to C₄) monoalkylamino groups, (C₁ to C₄) dialkylamino groups, (C₁ to C₄) trialkylammonium groups, (C₁ to C₄) monohydroxyalkylamino groups, imidazolinium, and —NH₃⁺.

Examples of (C₁ to C₄) monoalkylamino groups are —NHCH₃, —NHCH₂CH₃, —NHCH₂CH₂CH₃, —NHCH(CH₃)₂.

Examples of (C₁ to C₄) dialkylamino group are —N(CH₃)₂, —N(CH₂CH₃)₂.

Examples of (C₁ to C₄) alkoxy-(C₁ to C₄) alkyl groups are the groups —CH₂CH₂—O—CH₃, —CH₂CH₂CH₂—O—CH₃, —CH₂CH₂—O—CH₂CH₃, —CH₂CH₂CH₂—O—CH₂CH₃, —CH₂CH₂—O—CH(CH₃)₂, —CH₂CH₂CH₂—O—CH(CH₃)₂.

Examples of (C₁ to C₄) alkoxy-(C₁ to C₄) alkoxy groups are the groups —O—CH₂CH₂—O—CH₃, —O—CH₂CH₂CH₂—O—CH₃, —O—CH₂CH₂—O—CH₂CH₃, —O—CH₂CH₂CH₂—O—CH₂CH₃, —O—CH₂CH₂—O—CH(CH₃)₂, —O—CH₂CH₂CH₂—O—CH(CH₃)₂.

Examples of hydroxy-(C₁ to C₄) alkoxy residues are —O—CH₂OH, —O—CH₂CH₂OH, —O—CH₂CH₂CH₂OH, —O—CH₂CH(OH)CH₃, —O—CH₂CH₂CH₂CH₂OH.

Examples of (C₁ to C₄) aminoalkyl residues are —CH₂NH₂, —CH₂CH₂NH₂, —CH₂CH₂CH₂NH₂, —CH₂CH(NH₂)CH₃, —CH₂CH₂CH₂CH₂NH₂.

An example of aryl groups is the phenyl group, which can also be substituted.

Examples of aryl-(C₁ to C₄) alkyl groups are the benzyl group and the 2-phenylethyl group.

It is preferred in the context of all the oxidation dye precursors recited (in particular those mentioned as preferred) to use one of the preferred particulate amorphous metal oxides (in particular one of the embodiments (A) to (F)) (see above), in particular in the preferred quantities or quantity ratios, for sorption of the oxidation dye precursor.

At least one anionic surfactant is suitable as a cosmetic active agent sorbed, in particular in order to furnish a powdered composition for cleaning keratinic fibers.

The powdered compositions contemplated herein contain anionic surfactants preferably in quantities from about 0.5 wt % to about 30.0 wt %, based on the total agent. Quantities from about 5.0 wt % to about 15.0 wt % are particularly preferred.

The following preferred anionic surface-active substances are suitable as anionic surfactants in compositions contemplated herein:
linear and branched fatty acids having 8 to 30 carbon atoms (soaps),
ether carboxylic acids of the formula R—O—(CH₂—CH₂O)ₓ—CH₂—COOH, in which R is a linear alkyl group having 8 to 30 carbon atoms and x=0 or is 1 to 16, and salts thereof,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic acid mono- and dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups,
linear alkanesulfonates having 8 to 24 carbon atoms;
linear alpha-olefinsulfonates having 8 to 24 carbon atoms,
alpha-sulfo fatty acid methyl esters of fatty acids having 8 to 30 carbon atoms,
alkyl sulfates and alkyl polyglycol ether sulfates of the formula R—O(CH₂—CH₂—O)ₓ—OSO₃H, in which R is a preferably linear alkyl group having 8 to 30 carbon atoms and x=0 or is 1 to 12,
hydroxysulfonates corresponding to at least one of the two following formulas, or mixtures thereof, as well as salts thereof:

CH₃—(CH₂)_{y}—CHOH—(CH₂)ₚ—(CH—SO₃M)-(CH₂)_{z}—CH₂—O—(CₙH₂ₙO)ₓ—H, and/or

CH₃—(CH₂)_{y}—(CH—SO₃M)-(CH₂)ₚ—CHOH—(CH₂)_{z}—CH₂—O—(CₙH₂ₙO)ₓ—H,
wherein in both formulas y and z=0 or are integers from 1 to 18, p=0, 1, or 2, and the sum (y+z+p) is a number from 12 to 18, x=0 or is a number from 1 to 30, and n is an integer from 2 to 4, and M=hydrogen or alkali, in particular sodium, potassium, lithium, alkaline earth, in particular magnesium, calcium, zinc, and/or an ammonium ion, which optionally can be substituted, in particular mono-, di-, tri- or tetraammonium ions having C1 to C4 alkyl, alkenyl, or aryl residues,
sulfated hydroxyalkylpolyethylene glycol ethers and/or hydroxyalkylenepropylene glycol ethers of the formula R¹—(CHOSO₃M)-CHR³—(OCHR⁴—CH₂)n-OR², where R¹ denotes a linear alkyl residue having 1 to 24 carbon atoms, R² a linear or branched, saturated alkyl residue having 1 to 24 carbon atoms, R³ denotes hydrogen or a linear alkyl residue having 1 to 24 carbon atoms, R⁴ denotes hydrogen or a methyl residue, and M denotes hydrogen, ammonium, alkylammonium, alkanolammonium, in which the alkyl and alkanol residues each comprise 1 to 4 carbon atoms, or a metal atom selected from lithium, sodium, potassium, calcium, or magnesium, and n denotes a number in the range from 0 to 12, and furthermore the total number of carbon atoms contained in R¹ and R³ is 2 to 44,
sulfonates of unsaturated fatty acids having 8 to 24 carbon atoms and 1 to 6 double bonds,
esters of tartaric acid and citric acid with alcohols that represent addition products of approximately 2 to 15 molecules of ethylene oxide and/or propylene oxide with fatty alcohols having 8 to 22 carbon atoms,
alkyl and/or alkenyl ether phosphates of the formula

R¹(OCH₂CH₂)ₙ—O(PO—OX)—OR²
in which R¹ preferably denotes an aliphatic hydrocarbon residue having 8 to 30 carbon atoms, R² denotes hydrogen, a (CH₂CH₂O)ₙR² residue, or X, n denotes numbers from 1 to 10, and X denotes hydrogen, an alkali or alkaline earth metal, or NR³R⁴R⁵R⁶, where R³ to R⁶ mutually independently denote hydrogen or a C₁ to C₄ hydrocarbon residue,
sulfated fatty acid alkylene glycol esters of the formula

RCO(AlkO)ₙSO₃M,

in which RCO— denotes a linear or branched, aliphatic, saturated and/or unsaturated acyl residue having 6 to 22 carbon atoms, Alk denotes CH₂CH₂, CHCH₃CH₂, and/or CH₂CHCH₃, n denotes numbers from 0.5 to 5, and M denotes a metal, such as an alkali metal, in particular sodium, potassium, lithium, an alkaline earth metal, in particular magnesium, calcium, zinc, or an ammonium ion such as ⁺NR³N⁴N⁵N⁶, where R³ to R⁶ mutually independently denote hydrogen or a C₁ to C₄ hydrocarbon residue,
monoglyceride sulfates and monoglyceride ether sulfates of the formula R⁸OC—(OCH₂CH₂)ₓ—OCH₂—[CHO(CH₂CH₂O)_{y}H]—CH₂O(CH₂CH₂O)_{z}—SO₃X,
in which R⁸CO denotes a linear or branched acyl residue having 6 to 22 carbon atoms, x, y, and z in total denote 0 or numbers from 1 to 30, preferably 2 to 10, and X denotes an alkali or alkaline earth metal. Typical examples of monoglyceride (ether) sulfates suitable for purposes herein are the reaction products of lauric acid monoglyceride, coconut fatty acid monoglyceride, palmitic acid monoglyceride, stearic acid monoglyceride, oleic acid monoglyceride, and tallow fatty acid monoglyceride, and ethylene oxide adducts thereof with sulfur trioxide or chlorosulfonic acid in the form of sodium salts thereof. It is preferable to use monoglyceride sulfates in which R⁸CO denotes a linear acyl residue having 8 to 18 carbon atoms,
amide ether carboxylic acids, R¹—CO—NR²—CH₂CH₂—O—(CH₂CH₂O)ₙCH₂COOM, where R¹ is a straight-chain or branched alkyl or alkenyl residue having a number of carbon atoms in the chain from 2 to 30, n denotes an integer from 1 to 20, and R² denotes hydrogen, a methyl, ethyl, propyl, isopropyl, n-butyl, tert-butyl, or isobutyl residue and M denotes hydrogen or a metal, such as an alkali metal, in particular sodium, potassium, lithium, an alkaline earth metal, in particular magnesium, calcium, zinc, or an ammonium ion, such as ⁺NR³R⁴R⁵R⁶, where R³ to R⁶ mutually independently denote hydrogen or a C1 to C4 hydrocarbon residue. Products of this kind are obtainable, for example, from the Chem-Y company under the product designation Akypo®.
Acyl glutamates of the formula XOOC—CH2CH2CH(C(NH)OR)—COOX, in which RCO denotes a linear or branched acyl residue having 6 to 22 carbon atoms and 0 and/or 1, 2, or 3 double bonds, and X denotes hydrogen, an alkali metal and/or alkaline earth metal, ammonium, alkylammonium, alkanolammonium, or glucammonium,
condensation products of a water-soluble salt of a water-soluble protein hydrolysate with a C8 to C30 fatty acid. Such products have been commercially obtainable for some time under the trade names Lamepon®, Maypon®, Gluadin®, Hostapon® KCG, or Amisoft®,
alkyl- and/or alkenyloligoglycoside carboxylates, sulfates, phosphates, and/or isethionates,
acyl lactylates, and
hydroxy mixed ether sulfates.

If the anionic surfactants used (in particular mild anionic surfactants) comprise polyglycol ether chains, it is very particularly preferred that they exhibit a restricted homolog distribution. It is further preferred in the case of mild anionic surfactants having polyglycol ether units that the number of glycol ether groups be equal to 1 to 20, preferably 2 to 15, particularly preferably 2 to 12. Particularly mild anionic surfactants having polyglycol ether groups without a restricted homolog distribution can also be obtained, for example, if on the one hand the number of polyglycol ether groups is equal to 4 to 12, and Zn ions or Mg ions are selected as a counter ion. One example thereof is the commercial product Texapon® ASV.

It is preferred in the context of all the anionic surfactants recited (in particular those mentioned as preferred) to use one of the preferred particulate amorphous metal oxides (in particular one of the embodiments (A) to (F)) (see above), in particular in the preferred quantities or quantity ratios, for sorption of the anionic surfactant.

At least one keratin-reducing compound is suitable as a cosmetic active agent sorbed, in particular in order to furnish a powdered composition for the permanent deformation of keratinic fibers.

The permanent deformation of keratin fibers is usually carried out in such a way that the fibers are treated with a keratin-reducing compound and shaped by means of mechanical deformation aids. After a contact time a rinse is performed with water or an aqueous solution. In a second step, the fibers are then treated with a preparation of an oxidizing agent. After a contact time this, too, is rinsed out, and the fibers are released from the mechanical deformation aids (rollers, curlers).

The keratin-reducing compound cleaves a portion of the disulfide bonds of the keratin to yield —SH groups, thus resulting in a loosening of the peptide crosslink network and, because the tension of the fiber due to the mechanical deformation, in a reorientation of the keratin structure. Disulfide bonds are re-linked under the influence of the oxidizing agent, and the keratin structure is thereby re-immobilized in the intended deformation. Permanent wave treatment of human hair represents one known method of this kind. This can be applied both to generate curls and waves in straight hair, and to straighten frizzy hair.

The keratin-reducing compounds contained in sorbed form in the powdered composition are preferably selected from compounds having at least one thiol group and derivatives thereof, sulfites, hydrogen sulfites, disulfites.

Compounds having at least one thiol group, and derivatives thereof, are, for example, thioglycolic acid, thiolactic acid, thiomalic acid, phenylthioglycolic acid, mercaptoethanesulfonic acid, and salts and esters thereof (for example, isooctyl thioglycolate and isopropyl thioglycolate), cysteamine, cysteine, Bunte salts, and salts of sulfurous acid. The monoethanolammonium salts or ammonium salts of thioglycolic acid and/or of thiolactic acid, and the free acids, are preferably suitable. These are employed in the powdered preparation preferably in concentrations from about 0.5 to about 2.0 mol/kg.

Examples of keratin-reducing compounds of the disulfites that can be contained in sorbed fashion in the powdered composition are alkali disulfites such as sodium disulfite (Na₂S₂O₅) and potassium disulfite (K₂S₂O₅) as well as magnesium disulfite and ammonium disulfite ((NH₄)₂S₂O₅). Ammonium disulfite is preferred. Examples of keratin-reducing compounds of the hydrogen sulfites that can be contained in sorbed fashion in the powdered composition are hydrogen sulfites as an alkali, magnesium, ammonium, or alkanolammonium salt based on a C₂ to C₄ mono-, di, or trialkanolamine Ammonium hydrogen sulfite can be a particularly preferred hydrogen sulfite. Examples of keratin-reducing compounds of the sulfites that can be contained in sorbed fashion in the powdered composition are sulfites as an alkali, ammonium, or alkanolammonium salt based on a C₂ to C₄ mono-, di-, or trialkanolamine Ammonium sulfite is preferred.

Preferred sulfur-containing keratin-reducing compounds are cysteamine, cysteine, Bunte salts, and salts of sulfurous acid, thioglycolic acid, thiolactic acid, thiomalic acid, mercaptoethanesulfonic acid, and salts and esters of the aforesaid thio acids.

The keratin-reducing compound is employed preferably in a quantity from about 5 to about 20 wt %, based on the total powdered composition.

It is preferred in the context of all the keratin-reducing compounds recited (in particular those mentioned as preferred) to use one of the preferred particulate amorphous metal oxides (in particular one of the embodiments (A) to (F)) (see above), in particular in the preferred quantities or quantity ratios, for sorption of the keratin-reducing compound.

It has proven to be particularly advantageous in the context of all the aforesaid embodiments, in particular for use of the powdered compositions contemplated herein on a dry substrate, to additionally add core-shell particles, whose shell contains particles of at least one hydrophobized metal oxide powder and whose core comprises a liquid aqueous phase, to the powdered composition.

The core-shell particles of the powdered composition in accordance with the above preferred embodiment comprise a core that comprises an aqueous liquid phase. The core is thus present in liquid form. Surrounding this core is a shell that is based on separable individual particles of at least one hydrophobized metal oxide powder.

The nature of the hydrophobized metal oxide is in principle not limited, provided there is assurance that a corresponding core-shell particle is produced upon intensive mixing with the liquid aqueous phase. Those metal oxides which have been modified, at least on the surface of the particles, in such a way that the modified particles are wetted less by water than the unmodified particle, are to be understood as “hydrophobized” for purposes herein. Silanized hydrophobized metal oxides are particularly preferred. At least one representative of the group that is constituted from silanes, halosilanes, alkoxysilanes, and silazanes is preferably suitable as a reagent for silanizing the metal oxide.

Preferably suitable hydrophobized metal oxides of the hydrophobized metal oxide powder are selected from at least one representative of the group that is constituted from hydrophobized silicates, hydrophobized aluminum silicates, hydrophobized titanium dioxide, and hydrophobized silicon dioxide.

Particularly preferred aluminum silicates (also called “aluminosilicates”) are selected from phyllosilicates, tectosilicates, or mixtures thereof

Preferably suitable phyllosilicates are selected from kaolins (in this case in particular from kaolinite, dickite, hallosite, and nacrite), serpentine, talc, pyrophyllite, montmorillonite, quartz, bentonite, mica (in this case in particular from illite, muscovite, paragonite, phlogopite, biotite, lepidolite, margarite, smectite (in this case in particular from montmorillonite, saponite, nontronite, hectorite)).

Preferably suitable tectosilicates are selected from feldspar minerals (in particular albite, orthoclase, anorthite, leucite, sodalite, hauyne, labradorite, lazurite, nosean, nepheline), zeolites.

The core-shell particles of the powdered compositions preferred herein contain the hydrophobized metal oxide powder preferably in a quantity from about 0.5 to about 30 wt %, based on the weight of the core-shell particles contained in the powdered composition.

It has furthermore proven to be preferred if the hydrophobized metal oxides have a particle diameter of less than about 5 μm, preferably less than about 1 μm, particularly preferably between about 20 and about 100 nm.

Particularly preferably, the powdered composition contemplated herein contains as a hydrophobized metal oxide powder of the core-shell particles at least one hydrophobized silicon dioxide, in particular at least one silanized hydrophobized silicon dioxide.

At least one representative of the group that is constituted from silanes, halosilanes, alkoxysilanes, and silazanes is preferred as a reagent for silanizing the silicon dioxide.

Preferred representatives of the group of silanes are hexa-(C₁ to C₂₀) alkyldisilanes, in particular hexamethyldisilane.

If a halosilane is utilized as a silylating agent, the preferred halosilane selected is at least one compound from the group that is constituted from the compounds

[(C₁-C₂₀)alkyl]_{z′}SiX_{(4-z′)}

X₃Si[(CH₂)ₙ—R]

X₂[(C₁-C₂₀)alkyl]Si(CH₂)ₙ—R

[(C₁-C₂₀)alkyl]_{(y′+1)}[R—(CH₂)ₙ]_{(2-y′)}SiX

in which
X signifies a chlorine, bromine, or iodine atom,
z′ is a number 1, 2, or 3,
y′ is a number 0, 1, or 2,
n is an integer from 1 to 20, and
R denotes a residue from
(C₁ to C₁₀) alkyl, aryl, (C₁ to C₆) perfluoroalkyl, —NH₂, —N₃, —SCN, —CH═CH₂,

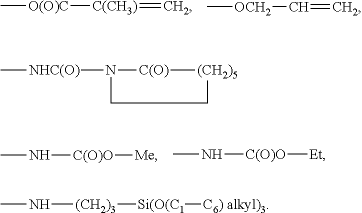

If an alkoxysilane is utilized as a silylating agent, the preferred alkoxysilane selected is at least one compound from the group that is constituted from the compounds

[(C₁-C₂₀)alkylO]_{z}Si(C₁-C₂₀)alkyl_{(4-z)}

[(C₁-C₂₀)alkylO]_{z}Si[(CH₂)ₙ—R]_{(4-z)}

[(C₁-C₂₀)alkylO]₂[(C₁-C₂₀)alkyl]Si(CH₂)ₙ—R

[(C₁-C₂₀)alkylO][(C₁-C₂₀)alkyl]₂Si(CH₂)ₙ—R

[(C₁-C₂₀)alkylO][(C₁-C₂₀)alkyl]Si(CH₂)ₙ—R₂

(C₁-C₂₀alkyl)₃SiO—C(CH₃)═N—Si(C₁-C₂₀)alkyl₃

in which
n is an integer from 1 to 20 and
z signifies a number 1, 2, or 3
R denotes a residue from
(C₁ to C₂₀) alkyl, aryl, (C₁ to C₆) perfluoroalkyl, —NH₂, —N₃, —SCN, —CH═CH₂,

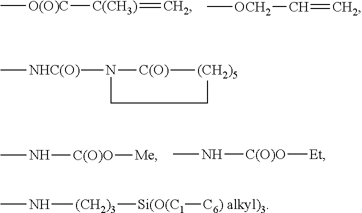

At least one compound from the class of disilazanes, in particular at least one compound from disilazanes of the formula

R′₂R″Si—NH—SiR′₂R″

in which
R′ signifies a (C₁ to C₂₀) alkyl group, and
R″ signifies a (C₁ to C₂₀) alkyl group or a vinyl group, is preferably selected as a preferred silazane. A particularly preferred silazane is hexamethyldisilazane.

All the aforementioned alkyl groups, whether (C₁ to C₆) alkyl, (C₁ to C₁₀) alkyl, or (C₁ to C₂₀) alkyl, can be both cyclic and linear or branched. Examples of alkyl groups usable herein are methyl, ethyl, n-propyl, isopropyl, n-butyl, cyclopentyl, cyclohexyl, n-decyl, lauryl, myristyl, cetyl, stearyl, isostearyl, and behenyl.

An example of an aryl group is the phenyl group.

Examples of a (C₁ to C₆) perfluoroalkyl group are trifluoromethyl, perfluoroethyl, perfluoropropyl, and perfluorohexyl.

Hydrophobized silicon dioxides that are obtained by silanization of pyrogenic silicon dioxide are preferably employed.

Silanized hydrophobized silicon dioxides are particularly preferably selected from at least one compound of the group that is constituted from trimethyl silylate-coated silicon dioxide, dimethyl silylate-coated silicon dioxide, silicon dioxide, octyl silylate-coated silicon dioxide.

It is preferred in turn to select the hydrophobized metal oxide powder of the core-shell particles from silica silylates. These are hydrophobized silicon dioxides that conform to the INCI name Silica Silylate.

Those hydrophobized silicon dioxides which have a specific surface area according to BET between about 10 and about 400 m²/g, preferably between about 80 and about 300 m²/g, are preferred. Those hydrophobized silicon dioxides which are silanized, in particular Silica Silylate, are in turn preferably suitable.

A plurality of suitable hydrophobized silicon dioxides are commercially obtainable. Examples that can be recited are Aerosil® R104 V, Aerosil® R106, Aerosil® R202, Aerosil® R805, Aerosil® R812, Aerosil® R812S, Aerosil® R972, and Aerosil® R8200, all Degussa, and HDK® H2000, HDK® H2050, and HDK® H3004, all Wacker.

It is particularly preferred to use the hydrophobized silicon dioxides that are obtainable under the names Aerosil® R202, Aerosil® R812S, or Aerosil® R972. The silicon dioxide having the INCI name Silica Silylate, which is marketed by the Degussa company under the name Aerosil® R812S, is very particularly preferably used.

The core-shell particles of the powdered compositions contemplated herein contain the hydrophobized silicon oxide powder preferably in a quantity from about 0.5 to about 30 wt %, based on the weight of the core-shell particles contained in the powdered composition. It is particularly preferred in turn to select the hydrophobized silicon dioxide powder from Silica Silylate, and to use it in a quantity from about 0.5 to about 30 wt %, based on the weight of the core-shell particles contained in the powdered composition. The optimum quantity here depends chiefly on the hydrophobicity of the silicon dioxide powder used. The more hydrophobic the silicon dioxide powder, the less thereof is needed in order to obtain a stable powdered product.

The powdered composition contemplated herein preferably contains as a hydrophobized metal oxide at least one hydrophobized phyllosilicate, in particular at least one hydrophobized mica and/or at least one hydrophobized talc. At least one silanized hydrophobized phyllosilicate or at least one silanized hydrophobized mica, and/or at least one hydrophobized talc, is in turn preferably suitable.

At least one representative of the group that is constituted from silanes, halosilanes, alkoxysilanes, and silazanes is preferably suitable as a reagent for silanizing the phyllosilicate or mica. For the particularly preferred reagents, reference is made, mutatis mutandis, to the statements regarding the silanization of silicon dioxide (see above).

Silanized hydrophobized micas are selected particularly preferably from at least one compound of the group that is constituted from trimethyl silylate-coated mica, dimethyl silylate-coated mica, octyl silylate-coated mica.

Silanized hydrophobized talc is selected particularly preferably from at least one compound of the group that is constituted from trimethyl silylate-coated talc, dimethyl silylate-coated talc, octyl silylate-coated talc.

Hydrophobically modified phyllosilicates usable herein that may be recited are, for example, hydrophobized mica silanized with triethoxycaprylsilane, hydrophobized mica silanized with triethoxycaprylsilane, for example from the LCW company.

The core-shell particles of the powdered compositions contemplated herein furthermore contain, as hydrophobized metal oxide powders, metal oxide particles coated at the surface with fluorine-containing organic groups (in particular, mica coated at the surface with fluorine-containing organic groups, titanium dioxide coated at the surface with fluorine-containing organic groups, silicon dioxide coated at the surface with fluorine-containing organic groups). Metal oxide particles coated at the surface with fluorine-containing organic groups which are particularly suitable for embodying this embodiment are those which have been coated at the surface with perfluorinated organic compounds, in particular with perfluoroalkyl residues. Said metal oxides that have been hydrophobized with at least one residue selected from perfluoroalkylsilyl, polyperfluoro-(C₂ to C₆) alkylene oxide, polysiloxane having perfluoroalkyl groups, perfluoroalkyl phosphate, polyfluoroalkyl phosphate ethers are in turn preferred here. Among these, those metal oxide particles coated at the surface with fluorine-containing organic groups which carry polyperfluoro-(C₂ to C₆) alkylene oxide residues at the surface are preferably used. These are preferably selected from mica coated with polyperfluoro-(C₂ to C₆) alkylene oxide and/or titanium dioxide coated with polyperfluoro-(C₂ to C₆) alkylene oxide and/or silicon dioxide coated with polyperfluoro-(C₂ to C₆) alkylene oxide and/or talc coated with polyperfluoro-(C₂ to C₆) alkylene oxide.

The commercial products PW Covafluor® (LCW company), PFS Talc JA 46-R® (Daito), Talc JA R46PF® (LCW), Submica M® (LCW) are particularly preferably usable in the context of this embodiment.

Very particularly preferably suitable in the context of this embodiment are those metal oxide particles coated at the surface with fluorine-containing organic groups which have been coated at the surface by reaction with at least one reagent of formula (I)

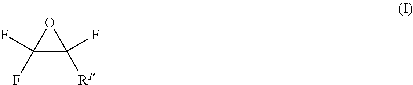

in which R^{F} denotes a perfluoro-(C₁ to C₄) alkyl group, in particular trifluoromethyl.

Metal oxide particles that have been coated with reagents of formula (I) acquire polyperfluoro-(C₂ to C₆) alkylene oxide residues bound covalently at the surface.

It is furthermore preferred in the context of the aforesaid embodiment if the metal oxide particles coated at the surface with fluorine-containing organic groups are coated with at least one compound of formula (Ia)

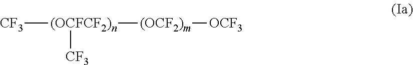

in which the sum n+m denotes an integer from 20 to 80. These compounds possess the INCI name Polyperfluoromethylisopropyl Ether. Compounds of formula (Ia) that have a molecular weight from 1500 to 4000 g/mol are particularly preferably suitable.

The commercial product PW F-MS (nanoscale titanium dioxide coated with polyperfluoromethylisopropyl ether (CAS no. 69991-67-9, EINECS 274-225-4) and triethoxycaprylylsilane (INCI name: CI 77891, Polyperfluoromethylisopropyl Ether, Triethoxycaprylylsilane)) of the Sensient/LCW company is very particularly preferably usable.

A “liquid aqueous phase” is understood as a liquid that contains at least about 40 wt %, in particular at least about 65 wt % water, based on the weight of the liquid aqueous phase in the core.

The liquid aqueous phase of the core-shell particles of the preferred embodiment of the powdered composition preferably contains an aqueous solvent selected from water or a mixture of water and a C₁ to C₄ alcohol, in particular ethanol. However, because surface-active substances and alcohols can in some circumstances wet hydrophobized silicon dioxide and thus negatively influence the hydrophobic properties, depending on the nature of the hydrophobized silicone dioxide used it can be necessary to keep the C₁ to C₄ alcohol content in the aqueous solvent below a critical maximum amount.

The core-shell particles in the liquid aqueous phase preferably contain, based on the weight of the core-shell particles of the powdered composition, about 70 wt % to about 90 wt %, particularly preferably about 80 to about 90 wt %, of an aqueous solvent.

Water, or a mixture of water and a maximum of about 60 wt % C₁ to C₄ alcohol, based on the solvent mixture, is therefore preferably used as an aqueous solvent. Particularly preferred aqueous solvents are water or a mixture of water and a maximum of about 30 wt % C₁ to C₄ alcohol, based on the solvent mixture. Very particularly preferably, water is used as an aqueous solvent.

The core-shell particles of the preferred embodiment of the powdered composition are suitable for releasing the liquid aqueous phase of the core from the core-shell particles by means of a mechanical load on the core-shell particles, in particular by friction and/or pressure, and thereby forming a liquid from the powdered composition.

If the powdered composition contemplated herein comprises said core-shell particles and is configured as an oxidative hair coloring agent comprising at least one oxidation dye precursor, the liquid aqueous phase of the core-shell particles preferably contains at least one oxidizing agent, in particular hydrogen peroxide.

If the powdered composition comprises said core-shell particles and is configured as an agent for permanent hair reshaping comprising at least one keratin-reducing compound, the liquid aqueous phase of the core-shell particles preferably contains at least one alkalizing agent Ammonia, alkali and ammonium carbonates and hydrogen carbonates, or organic amines such as monoethanolamine, and mixtures thereof, are considered preferred alkalizing agents.

The powdered compositions can be packaged in almost any containers. If the composition contains core-shell particles, all that is necessary is to ensure that the mechanical load on the powder upon removal of the composition is not so high that the powder is already converted into liquid form upon removal of the powder. Jars, bottles, or also Tetra Paks are suitable, for example; the container can be configured, for example, with a pouring and metering apparatus.

In a further embodiment, the powdered compositions contemplated herein can be made available by the following manufacturing method:

The particulate amorphous metal oxides containing at least one sorbed active agent are manufactured, for example, as follows: The aforesaid cosmetic active agent in liquid form (obtained either by dissolution in a solvent or by melting) is firstly slurried with the particulate amorphous metal oxide in a mixer (e.g. a propeller mixer), and then stirred until the particulate amorphous metal oxide has absorbed the aforesaid cosmetic active agent and a powdered composition results. Another manufacturing method is intimate blending of a solid sorbate with the aforesaid amorphous metal oxide as sorbent, e.g. in a mortar or mill.

The core-shell particles, as an addition in the context of the preferred embodiment of the powdered composition, can easily be manufactured. It has proven successful, when different metal oxide particles coated according to methods herein, firstly to mix all the metal oxide particles coated as described herein. Then, in a separate mixer, all the ingredients (except for the aforesaid metal oxide particles coated as described) are incorporated into the aqueous phase while stirring. Lastly, the coated metal oxide particles are added to the aqueous phase with intensive stirring. The mixing time required depends on the mixing energy introduced and on the respective composition of the mixture, but as a rule is between about 15 seconds and about 5 minutes. If mixing is too short, a stable powder does not form and formation of an oily phase occurs. If the mixing time is too long, the powder initially produced transitions into a doughy or creamy consistency; this process proceeds irreversibly. It is therefore recommended to ascertain the optimum mixing time for the particular system by means of a few preliminary experiments.

A powdered composition as contemplated herein comprising core-shell particles is furnished, for example, by carefully blending the core-shell particles with the aforesaid sorbate-containing powdered amorphous metal oxide.

An exemplary embodiment provides for the use of a powdered composition as contemplated herein for the cosmetic treatment of keratinic fibers, in particular human hair
for the temporary deformation of keratin-containing fibers, in particular human hair, or/and
for coloring keratin-containing fibers, in particular human hair, or/and
for cleaning keratin-containing fibers, in particular human hair, or/and
for conditioning keratin-containing fibers, in particular human hair, or/and
for permanent reshaping of keratin-containing fibers, in particular human hair.

It is in turn particularly preferred to use the powdered compositions for the temporary deformation of keratin-containing fibers, in particular human hair.

“Keratin-containing fibers” are to be understood herein as furs, wool, feather, and in particular human hair.

Another exemplary embodiment provides for a method for cosmetic treatment of keratin-containing fibers, in particular human hair, in which method
a) the keratin-containing fibers are optionally moistened,
b) a powdered composition as contemplated herein is applied onto the keratin-containing fibers,
c) if the powdered composition is one additionally containing said core-shell particles of the described herein, this powdered composition is exposed, during or after application onto the keratin-containing fibers, to a mechanical load, whereby the powdered composition comprising said core-shell particles converts into a liquid,
d) the applied composition is worked into the keratin-containing fibers and optionally rinsed out.

A method for the deformation of keratin-containing fibers, in particular human hair, in which method
a) the keratin-containing fibers are optionally moistened,
b) a powdered composition as contemplated herein is applied onto the keratin-containing fibers,
c) if the powdered composition is one additionally containing said core-shell particles described herein, this powdered composition is exposed, during or after application onto the keratin-containing fibers, to a mechanical load, whereby the powdered composition comprising said core-shell particles converts into a liquid,
d) the keratin-containing fibers are shaped
   before, after, or during application of the powdered composition and/or
   before, after, or during the mechanical load on the powdered composition,
e) the shape of the keratin-containing fibers is temporarily retained by the aforesaid powdered composition,
   is preferred.

It is preferred in the context of this embodiment of the method not to rinse the composition off the fibers (utilization as leave-on cosmetic).

For purposes of the aforesaid method, preferably firstly the desired quantity of powdered composition is removed from the container. The composition can be placed directly onto the keratinic fibers to be treated, or else, for example, onto one's hand. If the composition does not comprise any aforesaid core-shell particles (see above), the composition as contemplated herein is preferably used on moist to wet hair. If said core-shell particles are comprised, utilization on a dry fiber is also possible.

The powdered composition can be applied from the container directly onto the fibers, but of course also using an aid, for example one's hand, a paintbrush, a sponge, a cloth, a brush, or a comb. The aid can also be used to distribute the applied composition on the fibers.

If the powdered composition comprises said core-shell particles, the applied powder can be exposed to a mechanical load, for example by means of the hands or aids (see above), directly on the keratinic fibers, with the result that the liquid aqueous phase in the core-shell particles contained in the powdered composition becomes released directly on the fibers, and the effect of the sorbed active agent is produced. If the powdered composition comprising core-shell particles is firstly placed onto one's hand, it can then firstly be carefully distributed in the hair and in turn only then more strongly mechanically loaded, for example by deliberately massaging the powder into the hair. The result is that the liquid aqueous phase of the core-shell particles becomes released directly on the fibers, and the effect of the sorbed active agent is produced. An outstanding cosmetic effect can thereby be achieved in very controlled fashion. It is of course also possible to rub the powdered composition already on one's hand, and only then to apply the resulting liquid or pasty agent onto the keratinic fibers. This procedure is not preferred, however, since an essential advantage of the powdered consistency of the composition, namely good distribution capability, is thereby lost.

The Examples that follow are intended to explain the subject matter of the present invention without, however, limiting it in any way.

### Examples

Unless otherwise noted, the quantity indications indicated below are to be understood as percentages by weight.

| Raw material | E1 | E2 | E3 | E4 | E5 | E6 | E7 | E8 | E9 | E10 |
|---|---|---|---|---|---|---|---|---|---|---|
| Phenoxyethanol, pure | 0.6 | 0.6 | 0.6 | 1.0 | 1.0 | 0.6 | 0.6 | 0.6 | 0.6 | 0.6 |
| Water, demineralized | 6.4 | 6.4 | 6.4 | — | — | 6.4 | — | — | — | — |
| Isopropyl myristate | 5.0 | — | — | — | — | — | — | — | — | — |
| Wax emulsion | 7.0 | 7.0 | — | — | — | — | — | — | — | 7.0 |
| 2168/5BWCW | | | | | | | | | | |
| Sipernat ® 500LS | 10.0 | 25.0 | 25.0 | 24.0 | 20.0 | 22.5 | 22.0 | 21.0 | 18.5 | 16.4 |
| Aerosil ® R 812 S | 1.0 | — | — | — | — | — | — | — | — | — |
| Dow Corning ® | — | 5.0 | 5.0 | 5.0 | 5.0 | 5.0 | 5.0 | 5.0 | 5.0 | 5.0 |
| 193 C Fluid | | | | | | | | | | |
| Luviskol ® VA 64 W | — | — | — | — | 5.0 | — | — | — | — | — |
| Core-shell particles | — | — | — | — | — | 2.5 | 2.0 | 1.5 | 1.5 | 1.0 |
| PEG-45M | — | — | — | — | — | — | 0.4 | 2.0 | 2.0 | — |
| Sorbitol | — | — | — | — | — | — | 3.5 | 3.5 | 3.5 | — |
| Verityl AB-1000 Liquid | — | — | — | — | — | — | 2.0 | — | — | — |

| | |
|---|---|
| Glycerol | to 100 |

| Raw material | E11 | E12 | E13 | E14 | E15 | E16 | E17 | E18 | E19 | E20 |
|---|---|---|---|---|---|---|---|---|---|---|
| Phenoxyethanol, pure | 0.6 | 0.6 | 0.6 | 0.6 | 0.6 | 0.6 | 0.6 | 0.6 | 0.6 | 0.6 |
| Kahlwax 6240 | 10.0 | 10.0 | 5.0 | 5.0 | 5.0 | 5.0 | 10.0 | 10.0 | 10.0 | 5.0 |
| vegetable wax | | | | | | | | | | |
| Beeswax | — | 10.0 | — | — | — | — | — | — | — | — |
| Dow Corning 9701 | — | — | — | — | — | — | — | — | — | 2.0 |
| Cosmetic Powder | | | | | | | | | | |
| PEG-32 | 10.0 | 10.0 | 5.0 | 5.0 | 5.0 | 5.0 | 5.0 | 10.0 | 5.0 | — |
| PEG 8 | 10.0 | 15.0 | — | — | — | — | — | — | — | — |
| PEG-40 Hydrogenated | — | — | 5.0 | 5.0 | 5.0 | 5.0 | 5.0 | — | 5.0 | — |
| Castor Oil | | | | | | | | | | |
| Cocoglycerides + 7.3 EO | — | 15.0 | — | — | — | — | — | — | — | — |
| Sipernat ® 2200 | — | — | 14.4 | — | — | — | — | — | — | — |
| Sipernat ® 22 LS | — | — | — | 14.4 | — | — | — | — | — | — |
| Sipernat ® 50 | — | — | — | — | 14.4 | — | — | — | — | — |
| Sipernat ® 500LS | 23.0 | 23.0 | — | — | — | 25.0 | 23.0 | 23.0 | 23.0 | 22.4 |
| Wax emulsion | — | — | — | — | — | — | — | 7.0 | — | — |
| 2168/5BWCW | | | | | | | | | | |
| Polyvinylpyrrolidone | — | — | — | — | — | — | — | — | 1.4 | — |

| | |
|---|---|
| Glycerol | to 100 |

1. Manufacturing the Powdered Styling Agent

The powdered styling agents E1 to E19 contemplated herein were manufactured as described below.

All raw materials, except for Sipernat, Aerosil, and the Aerosil-containing mixture 1, were mixed with one another, the solid raw materials having been melted as necessary. The resulting mixture was blended with Sipernat and optionally Aerosil until the mixture had been sorbed by the Sipernat and optionally Aerosil, and a powdered composition resulted. The powdered mixture 1 was then (if present in the formula) carefully blended with the previously produced powder. The completed styling powder was introduced into polyethylene bottles.

2. Utilization

Nineteen hair strands were each treated with one of the styling agents recited above. The hair strands were moistened, and the agent was applied onto the moist strand and massaged in. The treated strand was laid out on a board, secured, and left to dry.

Outstanding hold was obtained for the compositions E1 to E20. The hair possessed appreciably more structure and texture. Even though particulate precipitated silicic acid was used, no visible matting of the hair was observable. The hair retained its natural shine.

3. List of Raw Materials Used

The raw materials employed in the context of the Examples are defined as follows:

| | |
|---|---|
| Amphomer ® | white powder; INCI name: Octylacrylamide/ |
| | Acrylates/Butylaminoethyl Methacrylate Copolymer |
| | (Akzo Nobel) |
| Sipernat ® 500 LS | precipitated silicic acid (white powder), pH (50 g/l |
| | suspension in water at 20° C.): 6.0; BET: 457 m²/g, |
| | DBP absorption: 325 g/100 g, Na₂O content: 0.6%, |
| | average particle diameter (d50): 4.5 μm, INCI name: |
| | Hydrated Silica |
| Sipernat ® 22 LS | precipitated silicic acid (white powder), pH (50 g/l |
| | suspension in water at 20° C.): 6.2; BET: 190 m²/g, |
| | DBP absorption: 265 g/100 g, Na₂O content: 1.0%, |
| | average particle diameter (d50): 4.5 μm, INCI name: |
| | Hydrated Silica |
| Sipernat ® 50 | precipitated silicic acid (white powder), pH (50 g/l |
| | suspension in water at 20° C.): 6.0; BET: 450 m²/g, |
| | DBP absorption: 335 g/100 g, Na₂O content: 0.6%, |
| | average particle diameter (d50): 40 μm, INCI name: |
| | Hydrated Silica |
| Sipernat ® 2200 | precipitated silicic acid (white powder), pH (50 g/l |
| | suspension in water at 20° C.): 6.0; BET: 185 m²/g, |
| | DBP absorption: 245 g/100 g, Na₂O content: 1.0%, |
| | average particle diameter (d50): 320 μm |
| Aerosil ® | modified pyrogenic silicic acid, INCI name: Silica |
| R 812 S | Silylate (Evonik Degussa) |
| Wax Emulsion | wax emulsion of beeswax and carnauba wax |
| 2168/5BWCW | |
| Core-shell particles | mixture of 20 wt % Aerosil R 812 S, 0.05 wt % |
| | Amphomer, 0.02 wt % citric acid, 0.2 wt % sodium |
| | benzoate, and 79.93 wt % water. Manufacture: |
| | Water, citric acid monohydrate, and sodium |
| | benzoate (and, for A2, PVP/VA 60/40 W NP) were |
| | mixed in a vessel. This resulted in a liquid having a |
| | pH between 4.5 and 5. The hydrophobized silicon |
| | dioxide powder Aerosil ® R 812 S (INCI name: |
| | Silica Silylate) was added to this liquid. After a |
| | stirring time respectively from 30 to 45 seconds, |
| | a stable powder had formed in each case. The |
| | Amphomer (if contained) was added as a solid |
| | to the stable powder, and blended in by stirring. |
| | The result is core-shell particles as defined by |
| | Claim 6, which liquefy upon mechanical loading. |
| DOW Corning 9701 | powdered (grain size <10 μm) mixture of a |
| Cosmetic Powder | crosslinked elastomeric silicone (INCI name: |
| | Dimethicone/Vinyl Dimethicone Crosspolmyer) and |
| | silica (INCI name: Silica) (Dow Corning) |
| DOW Corning ® | silicone-glycol copolymer (INCI name: PEG-12 |
| 193 C fluid | Dimethicone) (Dow Corning) |
| Cocoglycerides + | glycerides of coconut fatty acid cut, ethoxylated with |
| 7.3 EO | 7.3 equivalents ethylene oxide |
| Kahlwax ® | self-emulsifying wax ester (solid) |
| 6240 Vegetable Wax | (Kahl GmbH & Co.) |
| Verityl ® | 1,4-dihydroxymethylcyclohexane |
| AB 1000 Liquid | |
| Luviskol ® | copolymer of vinylpyrrolidone and vinyl acetate |
| VA 64 W | (60:40) (48-52% active substance in water, |
| | INCI name: VP/VA Copolymer) (BASF SE). |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A powdered composition comprising at least one particulate amorphous metal oxide, with the provision that at least one cosmetic active agent that is chosen from waxy esters, setting polymers, film-forming polymers, oxidation dye precursors, anionic surfactants, keratin-reducing compounds, and mixtures thereof is present in a manner sorbed in said metal oxide.

2. The powdered composition according to claim 1, wherein said metal oxide is a precipitated silicic acid that contains, based on its weight, in the range of from about 0.1 to about 1.0 wt % Na₂O.

3. The powdered composition according to claim 1, wherein said metal oxide has a DBP number from about 50 to about 800 g, per 100 g of metal oxide (measured per ASTM D 2414).

4. The powdered composition according to claim 1, wherein said metal oxide produces, in a 5-wt % slurry in distilled water, a pH of the resulting mixture from about 4.5 to about 8.0.

5. The powdered composition according to claim 1, wherein said metal oxide has a BET surface area from about 50 to about 1000 m²/g (measured in each case per ISO 5794-1).

6. The powdered composition according to claim 1, further comprising core-shell particles, whose shell contains particles of at least one hydrophobized metal oxide powder and whose core comprises a liquid aqueous phase.

7. The powdered composition according to claim 6, wherein a hydrophobized metal oxide of the hydrophobized metal oxide powder is chosen from hydrophobized silicates, hydrophobized aluminum silicates, hydrophobized titanium dioxide, and hydrophobized silicon dioxide.

8. The powdered composition according to claim 7, wherein the hydrophobized metal oxide powder is selected from silica silylate.

9. The powdered composition according to claim 6, wherein the core-shell particles contain in the liquid aqueous phase, based on the weight of the core-shell particles of the powdered composition, about 70 wt % to about 90 wt % of an aqueous solvent.

10. The powdered composition according claim 6, wherein the core-shell particles are suitable for releasing the liquid aqueous phase of the core from the core-shell particles by a mechanical load on the core-shell particles, and thereby for forming a liquid from the powdered composition.

11. The powdered composition according to claim 1, wherein said cosmetic active agent (or mixtures thereof) is present in a weight ratio range from about 1:1 to about 9:1, in a manner sorbed in the particulate amorphous metal oxide.

12. The powdered composition according to claim 1, wherein the powdered composition is suitable for the reshaping of keratin-containing fibers, and comprises as said cosmetic active agent at least one active agent that is selected from among waxy esters, setting polymers, film-forming polymers, and mixtures thereof.

13. (canceled)

14. A method for cosmetic treatment of keratin-containing fibers, in particular human hair, the method comprising the steps of:
a) optionally moistening the keratin-containing fibers;
b) applying a powdered composition onto the keratin-containing fibers, the powdered composition comprising: at least one particulate amorphous metal oxide, with the provision that at least one cosmetic active agent that is chosen from waxy esters, setting polymers, film-forming polymers, oxidation dye precursors, anionic surfactants, keratin-reducing compounds, and mixtures thereof is present in a manner sorbed in said metal oxide;
c) exposing the powdered composition, during or after application onto the keratin-containing fibers, to a mechanical load, if the powdered composition comprises core-shell particles whose shell contains particles of at least one hydrophobized metal oxide powder and whose core comprises a liquid aqueous phase, whereby the powdered composition converts into a liquid,
d) working the applied composition into the keratin-containing fibers and optionally rinsing the composition from the keratin-containing fibers.

15. The powdered composition according to claim 3, wherein said metal oxide has a DBP number from about 100 to about 500 g, per 100 g of metal oxide (measured per ASTM D 2414).

16. The powdered composition according to claim 15, wherein said metal oxide has a DBP number from about 200 to about 400 g, per 100 g of metal oxide (measured per ASTM D 2414).

17. The powdered composition according to claim 16, wherein said metal oxide has a DBP number from about 250 to about 350 g, per 100 g of metal oxide (measured per ASTM D 2414).

18. The powdered composition according to claim 4, wherein said metal oxide produces, in a 5-wt % slurry in distilled water, a pH of the resulting mixture from about 5.0 to 7.5.

19. The powdered composition according to claim 5, wherein said metal oxide has a BET surface area from about 100 to about 750 m²/g, (measured in each case per ISO 5794-1).

20. The powdered composition according claim 10, wherein the mechanical load is friction or pressure.
